(19) United States
(12) Patent Application Publication
(10) Pub. No.: US 2003/0207315 A1
(43) Pub. Date: Nov. 6, 2003

(54) Anti-aging nucleic acid and protein targets

(75) Inventors: Glenna C. Burmer, Seattle, WA (US); Joseph P. Brown, Seattle, WA (US); David K. Pritchard, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: LifeSpan BioSciences, Inc., Seattle, WA (US)

(21) Appl. No.: 10/454,565

(22) Filed: Jun. 3, 2003

Related U.S. Application Data
(63) Continuation of application No. 09/875,534, filed on Sep. 28, 2000, now abandoned.
(60) Provisional application No. 60/156,666, filed on Sep. 29, 1999.

(51) Int. Cl. C12Q 1/68

(52) U.S. Cl. 435/6

(57) ABSTRACT

This invention relates to the discovery of nucleic acids and proteins associated with aging processes. The identification of these aging-associated nucleic acids and proteins have diagnostic uses in detecting the aging status of a cell population as well as application for treating or delaying cellular and physiological changes that occur with aging or the onset of diseases typically associated with aging.

### CROSS-REFERENCE TO RELATED APPLICATIONS

This present application claims priority to U.S. Application No. 60/156,666 filed Sep. 29, 1999, which is herein incorporated by reference.

### FIELD OF THE INVENTION

This invention relates to the discovery of nucleic acids and proteins associated with aging processes. The identification of these aging-associated nucleic acids and proteins have diagnostic uses in detecting the aging status of a cell population as well as application for treating or delaying cellular and physiological changes that occur with aging or the onset of diseases typically associated with aging.

### BACKGROUND OF THE INVENTION

Aging is characterized by various changes in cellular and physiological processes and an increase in the incidence of age-related diseases such as cancer, heart disease, cataracts, neurodegenerative diseases, osteoporosis, arthritis, and hearing loss. Cellular changes that occur with aging include oxidation of lipids and proteins, damage to DNA, loss of proliferative capacity, telomere shortening, decline in RNA and protein synthesis, accumulation of cellular debris, and breakdown in cell to cell signaling. Physiological changes with aging include changes in skin tone, loss of muscle strength and muscle wasting, loss of hair and hair pigment, decreased elasticity of arteries, atherosclerosis, disrupted endocrine functions, loss of organ function, decreased metabolism and decline in the immune response.

The identification of protein and nucleic acid sequences that are differentially expressed in aging tissues, e.g, sequences that are associated with the loss of proliferative potential, has important diagnostic and therapeutic significance. For example, such sequences can be used as diagnostic markers or indicators to determine the aging status of tissues and can also serve as targets for intervention to slow or ameliorate the onset of age-related diseases or cellular and physiological changes associated with aging.

With respect to proliferative capacity of cells, normal human diploid cells have a finite potential for proliferative growth (Hayflick, L., et al., Exp. Cell Res. 25:585 (1961); Hayflick, L., Exp. Cell Res. 37:614 (1965)). Indeed, under controlled conditions, in vitro cultured human cells can maximally proliferate only to about 80 cumulative population doublings. The proliferative potential of such cells has been found to be a function of the number of cumulative population doublings which the cell has undergone (Hayflick, L., et al., Exp. Cell Res. 25:585 (1961); Hayflick, L., et al., Exp. Cell Res. 37: 614 (1985)). This potential is also inversely proportional to the in vivo age of the cell donor (Martin, G. M., et al., Lab. Invest. 23:86 (1979); Goldstein, S., et al., Proc. Natl. Acad. Sci. (U.S.A.) 64:155 (1969); Schneider, E. L., Proc. Natl. Acad. Sci. (U.S.A) 73:3584 (1976); LeGuilty, Y., et al., Gereontologia 19:303 (1973)).

Cells that have exhausted their potential for proliferative growth are said to have undergone “senescence.” Although a variety of theories have been proposed to explain the phenomenon of cellular senescence in vitro, experimental evidence suggests that the age-dependent loss of proliferative potential may be the function of a genetic program (Orgel, L. E., Proc. Natl. Acad. Sci. (U.S.A.) 49:517 (1963); De Mars, R., et al., Human Genet. 16:87 (1972); M. Buchwald, Mutat. Res. 44:401 (1977); Martin, G. M., et al., Amer. J. Pathol. 74:137 (1974); Smith, J. R., et al., Mech. Age. Dev. 13:387 (1980); Kirkwood, T. B. L., et al., Theor. Biol. 53:481 (1975). These genes associate with senescence may also include genes that are differentially expressed in aging tissue.

Two main strategies have been used to identify genes that are involved in the aging process. In relatively simple, genetically-tractable model organisms such as Saccharomyces cerevisae, Drosophila melanogaster, and C. elegans, genetic screens have been used to identify genes that, when mutated, confer enhanced longevity. In complex organisms, molecular screens have been used to identify genes that are differentially expressed during aging. For example, studies have compared expression patterns between young and senescent human diploid fibroblasts or between tissue sample derived from young versus old subjects. These studies have often identified different sets of genes and the results have been difficult to compare due to methodological differences, including variations in the methods used to derive cDNA libraries, cell cultures conditions, types of tissue used.

The present invention identifies aging-associated nucleic acid and protein sequences which are differentially expressed in multiple types of tissue. The high-density array technology employed herein has quantified the differential expression of 6,000 genes across multiple tissue types during aging. Thus, the present invention provides nucleic acid and protein sequences associated with aging and methods of identifying modulators of these sequences.

### SUMMARY OF THE INVENTION

The present invention provides isolated nucleic acids and proteins associated with aging. Such sequences can be used for diagnostic and therapetic purposes. For example, the sequences can be used to diagnose the aging status of tissues and in diagnosing tissues susceptible to age-related diseases. Moreover, the sequences can be used to treat cellular and phyiological changes that occur during aging. For example, drugs or modulators that alter either expression levels or the function of aging-associated differentially expressed genes can be used to slow aging-related changes or the development of age-associated diseases. Gene therapy can also be used to alter the expression levels of aging-associated genes or proteins. The ability to slow or modify aging-associated gene expression can, for example, lower the incidence and retard the progression of age-related diseases.

In one aspect, the present invention provides a method for identifying a modulator of expression of an age-associated gene, the method comprising: culturing the cell in the presence of the modulator to form a first cell culture; contacting RNA from the first cell culture with a probe which comprises a polynucleotide sequence associated with aging; determining whether the amount of the probe which hybridizes to the RNA from the first cell culture is increased or decreased relative to the amount of the probe which hybridizes to RNA from a second cell culture grown in the absence of the modulator, and further, the method can comprise detecting a phenotype indicative of altered aging properties in the cell population that is treated with the modulator. In one embodiment of this method, the probe comprises at least about 10 nucleotides from a polynucleotide sequence selected from the group consisting of the sequences set out in Table 1 which show increased expression with aging. In an alternative embodiment, the probe comprises at least about 10 nucleotides from a polynucleotide sequence selected from the group consisting of the sequences set out in Table 1 which show decreased expression with aging. In another embodiment, the method of identifying a modulator of expression of aging-associated genes can comprise the use of multiple probe, for example, using 1, 2, 3, 4, 5, 6, 7, 8, 9, 10, 15, 20, 25, 50, or more probes selected from the group consisting of the sequences set out in Table 1, to identify changes in expression of multiple aging-associated genes, and further, changes in a cellular phenotype associated with aging.

Altered cellular phenotypes associated with aging include, for example, a change in cellular morphology; a change in the proliferative potential of a cell, wherein an aged cell regains proliferative potential; a resumption of an aged cell's ability to respond to exogenous growth factors, a decrease in the oxidation of lipids and proteins, decreased damage to DNA, etc.

In still another aspect, the present invention provides kits for carrying out the various methods. For instance, in one embodiment, a kit is provided for detecting whether a cell is aging, the kit comprising: a probe which comprises a polynucleotide sequence associated with aging; and a label for detecting the presence of the probe. In one embodiment, the probe comprises at least about 10 nucleotides from a polynucleotide sequence selected from the group consisting of the sequences listed in Table 1. Additionally, this kit can further comprise a plurality of probes each of which comprises a polynucleotide sequence associated with aging; and a label or labels for detecting the presence of the plurality of probes. The probes can optionally be immobilized on a solid support (e.g., a chip).

The invention also includes the use of antisense methods for studying aging in animals and cells. Typically, an identified gene can be studied by knocking out the gene in an animal and observing the effect on the animal phenotype. Knockouts can be achieved by transposons which insert by homologous recombinations, antisense or ribozymes specifically directed at disturbing the embryonic stem cells of an organism such as a mouse. Ribozymes can include any of the various types of ribozymes modified to cleave the mRNA encoding, for example, the aging-associated protein. Examples include hairpins and hammerhead ribozymes. Finally, antisense molecules which selectively bind, for example, to the aging-assoicated protein mRNA are expressed via expression cassettes operably linked to subsequences of the aging-associated protein gene and generally comprise 20-50 base long sequences in opposite orientation to the mRNA to which they are targeted.

Similarly, the present invention provides a method for modulating the aging of a cell in a patient in need thereof, the method comprising administering to the patient a compound that modulates the aging of the cell. In one embodiment, the compound increases or decreases the expression level of a nucleic acid associated with aging. In this embodiment, the nucleic acid can, for example, comprise at least about 10 nucleotides from a polynucleotide sequence selected from the group consisting of the sequences set out in Table 1.

### DEFINITIONS

“Amplification” primers are oligonucleotides comprising either natural or analog nucleotides that can serve as the basis for the amplification of a select nucleic acid sequence. They include, for example, both polymerase chain reaction primers and ligase chain reaction oligonucleotides.

“Antibody” refers to a polypeptide substantially encoded by an immunoglobulin gene or immunoglobulin genes, or fragments thereof which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Fundamental Immunology, Third Edition, W. E. Paul, ed., Raven Press, N.Y. 1993). Wile various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies (e.g., single chain Fv).

“Associated” in the context of aging refers to the relationship of the relevant nucleic acids and their expression, or lack thereof, to aging in the subject cell. For example, aging can be associated with expression of a particular gene that is not expressed, or is expressed at a lower level, in a young, or non-aged, cell. Conversely, an aging-associated gene can be one that is not expressed in an aged cell, or is expressed at a lower level in the aged cell than in a non-aged cell. Frequently, a young phenotype is the phenotype observed in cells or tissues that are obtained from an individual of about 30 years or less in age, whereas an aged phenotype is the phenotype observed in cells or tissues that are obtained from an individual of about 65 years or less in age.

“Biological samples” refers to any tissue or liquid sample having genomic DNA or other nucleic acids (e.g., mRNA) or proteins. It includes both cells with a normal complement of chromosomes and cells with altered chromosomes relative to normal.

“Competent to discriminate between the wild type gene and the mutant form” means a hybridization probe or primer sequence that allows the trained artisan to detect the presence or absence of base changes, deletions or additions to the nucleotide sequence of interest. A probe sequence is a sequence containing the site that is changed, deleted or added to. A primer sequence will hybridize with the sequences surrounding or flanking the base changes, deletions or additions and, using the gene sequence as template, allow the further synthesis of nucleotide sequences that contain the base changes or additions. In addition, the probe may act as a primer. It is important to point out that this invention allows for the design of PCR primers capable of amplifying entire exons. To achieve this, primers need hybridize with intron sequences. This invention provides such intron sequences.

The term “gene” means the segment of DNA involved in producing a polypeptide chain; it includes regions preceding and following the coding region (leader and trailer) as well as intervening sequences (introns) between individual coding segments (exons).

A “heterologous sequence” or a “heterologous nucleic acid,” as used herein, is one that originates from a source foreign to the particular host cell, or, if from the same source, is modified from its original form. Thus, a heterologous gene associated with aging in a host cell includes an aging-associated gene that is endogenous to the particular host cell, but has been modified. Modification of the heterologous sequence may occur, e.g., by treating the DNA with a restriction enzyme to generate a DNA fragment that is capable of being operably linked to the promoter. Techniques such as site-directed mutagenesis are also useful for modifying a heterologous sequence.

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein which is the predominant species present in a preparation is substantially purified. In particular, an isolated gene is separated from open reading frames which flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

“Non-proliferating cells” are those which are said to be in a Gₒ-phase where the cells are in a resting stage of arrested growth at the Gₒ phase, usually because they are deprived of an essential nutrient and cannot grow exponentially.

The term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al., 1992; Rossolini et al., Mol. Cell. Probes 8:91-98 (1994)). The term nucleic acid is used interchangeably with gene, cDNA, and mRNA encoded by a gene.

“Nucleic acid derived from a gene” refers to a nucleic acid for whose synthesis the gene, or a subsequence thereof, has ultimately served as a template. Thus, an mRNA, a cDNA reverse transcribed from an mRNA, an RNA transcribed from that. cDNA, a DNA amplified from the cDNA, an RNA transcribed from the amplified DNA, etc., are all derived from the gene and detection of such derived products is indicative of the presence and/or abundance of the original gene and/or gene transcript in a sample.

As used herein a “nucleic acid probe” is defined as a nucleic acid capable of binding to a target nucleic acid (e.g., a nucleic acid associated with cell senescence or aging) of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions.

Nucleic acid probes can be DNA or RNA fragments. DNA fragments can be prepared, for example, by digesting plasmid DNA, or by use of PCR, or synthesized by either the phosphoramidite method described by Beaucage and Carruthers, Tetrahedron Lett. 22:1859-1862 (1981) (Beaucage and Carruthers), or by the triester method according to Matteucci, et al., J. Am. Chem. Soc., 103:3185 (1981) (Matteucci), both incorporated herein by reference A double stranded fragment may then be obtained, if desired, by annealing the chemically synthesized single strands together under appropriate conditions, or by synthesizing the complementary strand using DNA polymerase with an appropriate primer sequence. Where a specific sequence for a nucleic acid probe is given, it is understood that the complementary strand is also identified and included. The complementary strand will work equally well in situations where the target is a double-stranded nucleic acid.

A “labeled nucleic acid probe” is a nucleic acid probe that is bound, either covalently, through a linker, or through ionic, van der Waals or hydrogen bonds to a label such that the presence of the probe may be detected by detecting the presence of the label bound to the probe.

The term “target nucleic acid” refers to a nucleic acid (often derived from a biological sample) to which a nucleic acid probe is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding probe directed to the target. The term target nucleic acid may refer to the specific subsequence of a larger nucleic acid to which the probe is directed or to the overall sequence (e.g., gene or mRNA) whose expression level it is desired to detect. The difference in usage will be apparent from context.

The phrase “a nucleic acid sequence encoding” refers to a nucleic acid which contains sequence information for a structural RNA such as rRNA, a tRNA, or the primary amino acid sequence of a specific protein or peptide, or a binding site for a trans-acting regulatory agent. This phrase specifically encompasses degenerate codons (i.e., different codons which encode a single amino acid) of the native sequence or sequences which may be introduced to conform with codon preference in a specific host cell.

The term “operably linked” refers to functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence.

“Proliferating cells” are those which are actively undergoing cell division and grow exponentially.

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant expression cassette” or simply an “expression cassette” is a nucleic acid construct, generated recombinantly or synthetically, with nucleic acid elements that are capable of effecting expression of a structural gene in hosts compatible with such sequences. Expression cassettes include at least promoters and, optionally, transcription termination signals. Typically, the recombinant expression cassette includes a nucleic acid to be transcribed (e.g., a nucleic acid encoding a desired polypeptide), and a promoter. Additional factors necessary or helpful in effecting expression may also be used as described herein. For example, an expression cassette can also include nucleotide sequences that encode a signal sequence that directs secretion of an expressed protein from the host cell. Transcription termination signals, enhancers, and other nucleic acids that influence gene expression, can also be included in an expression cassette.

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

The phrase “substantially identical,” in the context of two nucleic acids or polypeptides, refers to two or more sequences or subsequences that have at least 60%, preferably 80%, most preferably 90-95% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see, generally, Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Another indication that two nucleic acids are substantially identical is that the two molecules hybridize to each other under stringent conditions. The phrase “hybridizing specifically to,” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

“Stringent hybridization conditions” and “stringent hybridization wash conditions” in the context of nucleic acid hybridization experiments, such as Southern and northern hybridizations, are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology—Hybridization with Nucleic Acid Probes, part I, chapter 2 “Overview of principles of hybridization and the strategy of nucleic acid probe assays,” Elsevier, N.Y. Generally, highly stringent hybridization and wash conditions are selected to be about 5° C. lower than the thermal melting point (Tₘ) for the specific sequence at a defined ionic strength and pH. Typically, under “stringent conditions,” a probe will hybridize to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2×SSC wash at 65° C. for 15 minutes (see, Sambrook, supra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2×(or higher) than that observed for an unrelated probe in the particular hybridization assay indicates detection of a specific hybridization. Nucleic acids which do not hybridize to each other under stringent conditions are still substantially identical if the polypeptides which they encode are substantially identical. This occurs, e.g., when a copy of a nucleic acid is created using the maximum codon degeneracy permitted by the genetic code.

A further indication that two nucleic acids or polypeptides are substantially identical is that the polypeptide encoded by the first nucleic acid is immunologically cross reactive with, or specifically binds to, the polypeptide encoded by the second nucleic acid. Thus, a polypeptide is typically substantially identical to a second polypeptide, for example, where the two peptides differ only by conservative substitutions.

The phrase “specifically (or selectively) binds to an antibody” or “specifically (or selectively) immunoreactive with”, when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised to the protein with the amino acid sequence encoded by any of the polynucleotides of the invention can be selected to obtain antibodies specifically immunoreactive with that protein and not with other proteins except for polymorphic variants. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See, Harlow and Lane (1988) Antibodies, A Laboratory Manual, Cold Spring Harbor Publications, New York (“Harlow and Lane”) for a description of immunoassay formats and conditions that can be used to determine specific immunoreactivity. Typically, a specific or selective reaction will be at least twice background signal or noise and more typically more than 10 to 100 times background.

A “conservative substitution,” when describing a protein, refers to a change in the amino acid composition of the protein that does not substantially alter the protein's activity. Thus, “conservatively modified variations” of a particular amino acid sequence refers to amino acid substitutions of those amino acids that are not critical for protein activity or substitution of amino acids with other amino acids having similar properties (e.g., acidic, basic, positively or negatively charged, polar or non-polar, etc.) such that the substitutions of even critical amino acids do not substantially alter activity. Conservative substitution tables providing functionally similar amino acids are well known in the art. See, also, Creighton (1984) Proteins, W. H. Freeman and Company. In addition, individual substitutions, deletions or additions which alter, add or delete a single amino acid or a small percentage of amino acids in an encoded sequence are also “conservatively modified variations”.

A “subsequence” refers to a sequence of nucleic acids or amino acids that comprise a part of a longer sequence of nucleic acids or amino acids (e.g., polypeptide) respectively.

### DETAILED DESCRIPTION AND PREFERRED EMBODIMENTS OF THE INVENTION

Age-related changes in cellular and physiological function are factors that influence the onset or progression of many of the diseases associated with human aging. Changes in cellular function with aging, including mitochondrial dysfunction, loss of proliferative capacity, and decreased protein synthesis, are associated with, or in some cases, result from alteration in gene expression.

The present invention provides nucleic acids and proteins that exhibit altered expression levels with aging and that regulate age-related changes. Host cells, vectors, and probes are described, as are antibodies to the proteins and uses of the proteins as antigens. The present invention provides methods for obtaining and expressing nucleic acids, methods for purifying gene products, other methods that can be used to detect and quantify the expression and quality of the gene product (e.g., proteins), and uses for both the nucleic acids and the gene products.

Cloning and Expression of the Nucleic Acids

A. General Recombinant DNA Methods.

This invention relies on routine techniques in the field of recombinant genetics. A basic text disclosing the general methods of use in this invention is Sambrook et al., Molecular Cloning, A Laboratory Manual, Cold Spring Harbor Publish., Cold Spring Harbor, N.Y. 2nd ed. (1989) and Kriegler, Gene Transfer and Expression: A Laboratory Manual, W. H. Freeman, N.Y., (1990), which are both incorporated herein by reference. Unless otherwise stated all enzymes are used in accordance with the manufacturer's instructions.

Nucleotide sizes are given in either kilobases (Kb) or base pairs (bp). These are estimates derived from agarose or acrylamide gel electrophoresis or, alternatively, from published DNA sequences.

Oligonucleotides that are not commercially available can be chemically synthesized according to the solid phase phosphoramidite triester method first described by S. L. Beaucage and M. H. Caruthers, Tetrahedron Letts., 22(20):1859-1862 (1981), using an automated synthesizer, as described in D. R. Needham Van Devanter et. al., Nucleic Acids Res., 12:6159-6168, 1984. Purification of oligonucleotides is, for example, by either native acrylamide gel electrophoresis or by anion-exchange HPLC as described in J. D. Pearson and F. E. Reanier, J. Chrom., 255:137-149, 1983.

The nucleic acids described here, or fragments thereof, can be used as a hybridization probe for a cDNA library to isolate the corresponding full length cDNA and to isolate other cDNAs which have a high sequence similarity to the gene or similar biological activity. Probes of this type preferably have at least 30 bases and may contain, for example, 50 or more bases. The probe may also be used to identify a cDNA clone corresponding to a full length transcript and a genomic clone or clones that contain the complete gene including regulatory and promotor regions, exons and introns. An example of such a screen includes isolating the coding region of the gene by using the known DNA sequence to synthesize an oligonucleotide probe. Labeled oligonucleotides having a sequence complementary to that of the nucleic acids of the present invention can be used to screen a library of human cDNA, genomic DNA or mRNA to determine which members of the library the probe hybridizes to.

The sequence of the cloned genes and synthetic oligonucleotides can be verified using the chemical degradation method of A. M. Maxam et al., Methods in Enzymology, 65:499560, (1980). The sequence can be confirmed after the assembly of the oligonucleotide fragments into the double-stranded DNA sequence using the method of Maxam and Gilbert, supra, or the chain termination method for sequencing double-stranded templates of R. B. Wallace et al., Gene, 16:21-26, 1981. Southern blot hybridization techniques can be carried out according to Southern et al., J. Mol. Biol., 98:503, 1975.

B. Cloning Methods for the Isolation of Nucleotide Sequences Encoding the Desired Proteins

In general, the nucleic acids encoding the subject proteins are cloned from DNA sequence libraries that are made to encode copy DNA (cDNA) or genomic DNA. The particular sequences can be located by hybridizing with an oligonucleotide probe, the sequence of which can be derived from the sequence listing provided herein, which provides a reference for PCR primers and defines suitable regions for isolating aging and senescent-associated specific probes. Alternatively, where the sequence is cloned into an expression library, the expressed recombinant protein can be detected immunologically with antisera or purified antibodies made against an aging-associated protein.

To make the cDNA library, one should choose a source that is rich in mRNA. The mRNA can then be made into cDNA, ligated into a recombinant vector, and transfected into a recombinant host for propagation, screening and cloning. Methods for making and screening cDNA libraries are well known. See, Gubler, U. and Hoffman, B. J., Gene 25:263-269, 1983 and Sambrook, supra.

For a genomic library, the DNA is extracted from the tissue and either mechanically sheared or enzymatically digested to yield fragments of preferably about 5-100 kb. The fragments are then separated by gradient centrifugation from undesired sizes and are constructed in bacteriophage lambda vectors. These vectors and phage are packaged in vitro, as described in Sambrook. Recombinant phage are analyzed by plaque hybridization as described in Benton and Davis, Science, 196:180-182 (1977). Colony hybridization is carried out as generally described in M. Grunstein et al., Proc. Natl. Acad. Sci. USA., 72:3961-3965 (1975).

An alternative method combines the use of synthetic oligonucleotide primers with polymerase extension on an mRNA or DNA template. This polymerase chain reaction (PCR) method amplifies nucleic acids of the protein directly from mRNA, from cDNA, from genomic libraries or cDNA libraries. Restriction endonuclease sites can be incorporated into the primers. Polymerase chain reaction or other in vitro amplification methods may also be useful, for example, to clone nucleic acids that code for proteins to be expressed, to make nucleic acids to use as probes for detecting the presence of mRNA encoding aging-related proteins in physiological samples, for nucleic acid sequencing, or for other purposes. U.S. Pat. Nos. 4,683,195 and 4,683,202 describe this method. Genes amplified by the PCR reaction can be purified from agarose gels and cloned into an appropriate vector.

Appropriate primers and probes for identifying the genes encoding aging-related protein from alternative mammalian tissues are generated from comparisons of the sequences provided herein. For a general overview of PCR, see PCR Protocols: A Guide to Methods and Applications. (Innis, M, Gelfand, D., Sninsky, J. and White, T., eds.), Academic Press, San Diego (1990), incorporated herein by reference.

Synthetic oligonucleotides can be used to construct genes. This is done using a series of overlapping oligonucleotides, usually 40-120 bp in length, representing both the sense and nonsense strands of the gene. These DNA fragments are then annealed, ligated and cloned.

The aging-associated gene is cloned using intermediate vectors before transformation into mammalian cells for expression. These intermediate vectors are typically prokaryote vectors or shuttle vectors. The proteins can be expressed in either prokaryotes or eukaryotes.

C. Expression in Prokaryotes

To obtain high level expression of a cloned gene, such as those cDNAs encoding aging-related proteins in a prokaryotic system, it is essential to construct expression plasmids which contain, at the minimum, a strong promoter to direct transcription, a ribosome binding site for translational initiation, and a transcription/translation terminator. Examples of regulatory regions suitable for this purpose in E. coli are the promoter and operator region of the E. coli tryptophan biosynthetic pathway as described by Yanofsky, C., J. Bacteriol., 158:1018-1024 (1984), and the leftward promoter of phage lambda (P_{L}) as described by Herskowitz, I. and Hagen, D., Ann. Rev. Genet., 14:399-445 (1980).

D. Expression in Eukaryotes

Standard eukaryotic transfection methods are used to produce mammalian, yeast or insect cell lines which express large quantities of the aging-associated protein which are then purified using standard techniques. See, e.g., Colley et al., J. Biol. Chem. 264:17619-17622, (1989), and Guide to Protein Purification, in Vol. 182 of Methods in Enzymology (Deutscher ed., 1990), both of which are incorporated herein by reference.

Transformations of eukaryotic cells are performed according to standard techniques as described by D. A. Morrison, J. Bact., 132:349-351 (1977), or by J. E. Clark-Curtiss and R. Curtiss, Methods in Enzymology, 101:347-362, Eds. R. Wu et. al., Academic Press, New York (1983).

Any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see Sambrook et al., supra). It is only necessary that the particular genetic engineering procedure utilized be capable of successfully introducing at least one gene into the host cell which is capable of expressing the protein.

The particular eukaryotic expression vector used to transport the genetic information into the cell is not particularly critical. Any of the conventional vectors used for expression in eukaryotic cells may be used. Expression vectors containing regulatory elements from eukaryotic viruses are typically used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

The vectors usually include selectable markers which result in gene amplification such as thymidine kinase, aminoglycoside phosphotransferase, hygromycin B phosphotransferase, xanthine-guanine phosphoribosyl transferase, CAD (carbamyl phosphate synthetase, aspartate transcarbamylase, and dihydroorotase), adenosine deaminase, dihydrofolate reductase, and asparagine synthetase and ouabain selection. Alternatively, high yield expression systems not involving gene amplification are also suitable, such as using a bacculovirus vector in insect cells, with a target protein encoding sequence under the direction of the polyhedrin promoter or other strong baculovirus promoters.

The expression vector of the present invention will typically contain both prokaryotic sequences that facilitate the cloning of the vector in bacteria as well as one or more eukaryotic transcription units that are expressed only in eukaryotic cells, such as mammalian cells. The vector may or may not comprise a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the transfected DNA integrates into the genome of the transfected cell, where the promoter directs expression of the desired gene. The expression vector is typically constructed from elements derived from different, well characterized viral or mammalian genes. For a general discussion of the expression of cloned genes in cultured mammalian cells, see, Sambrook et al., supra, Ch. 16.

The prokaryotic elements that are typically included in the mammalian expression vector include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells.

The expression vector contains a eukaryotic transcription unit or expression cassette that contains all the elements required for the expression of the aging-associated protein encoding DNA in eukaryotic cells. A typical expression cassette contains a promoter operably linked to the DNA sequence encoding the protein and signals required for efficient polyadenylation of the transcript. The DNA sequence encoding the protein may typically be linked to a cleavable signal peptide sequence to promote secretion of the encoded protein by the transformed cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus, the long term repeat from various retroviruses such as murine leukemia virus, murine or Rous sarcoma virus and HIV. See, Enhancers and Eukaryotic Expression, Cold Spring Harbor Pres, Cold Spring Harbor, N.Y. 1983, which is incorporated herein by reference.

In the construction of the expression cassette, the promoter is preferably positioned about the same distance from the heterologous transcription start site as it is from the transcription start site in its natural setting. As is known in the art, however, some variation in this distance can be accommodated without loss of promoter function.

In addition to a promoter sequence, the expression cassette should also contain a transcription termination region downstream of the structural gene to provide for efficient termination. The termination region may be obtained from the same gene as the promoter sequence or may be obtained from different genes.

If the mRNA encoded by the structural gene is to be efficiently translated, polyadenylation sequences are also commonly added to the vector construct. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40, or a partial genomic copy of a gene already resident on the expression vector.

In addition to the elements already described, the expression vector of the present invention may typically contain other specialized elements intended to increase the level of expression of cloned genes or to facilitate the identification of cells that carry the transfected DNA. For instance, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

1. Expression in Yeast

Synthesis of heterologous proteins in yeast is well known and described. Methods in Yeast Genetics, Sherman, F., et al., Cold Spring Harbor Laboratory, (1982) is a well recognized work describing the various methods available to produce aging-associated proteins in yeast.

For high level expression of a gene in yeast, it is essential to connect the gene to a strong promoter system as in the prokaryote and also to provide efficient transcription termination/polyadenylation sequences from a yeast gene. Examples of useful promoters include GAL1,1O (Johnson, M., and Davies, R. W., Mol. and Cell. Biol., 4:1440-1448 (1984)) ADH2 (Russell, D., et al., J. Biol. Chem., 258:2674-2682, (1983)), PHO5 (EMBO J. 6:675-680, (1982)), and MFα1. A multicopy plasmid with a selective marker such as Leu-2, URA-3, Trp-1, and His-3 is also desirable.

The MFα1 promoter is preferred for expression of the subject protein in yeast. The MFα1 promoter, in a host of the α mating-type, is constitutive, but is switched off in diploids or cells with the α mating-type. It can, however, be regulated by raising or lowering the temperature in hosts which have a ts mutation at one of the SIR loci. The effect of such a mutation at 35° C. on an α-type cell is to turn on the normally silent gene coding for the α mating-type. The expression of the silent α mating-type gene, in turn, turns off the MFα1 promoter. Lowering the temperature of growth to 27° C. reverses the whole process, i.e., turns the α mating-type off and turns the MFα1 on (Herskowitz, I. and Oshima, Y., in The Molecular Biology of the Yeast Saccharomyces, (eds. Strathern, J. N. Jones, E. W., and Broach, J. R., Cold Spring Harbor Lab., Cold Spring Harbor, N.Y., pp.181-209 (1982)).

The polyadenylation sequences are provided by the 3′-end sequences of any of the highly expressed genes, like ADH1, MFα1, or TPI (Alber, T. and Kawasaki, G., J. of Mol. & Appl. Genet. 1:419-434 (1982)).

A number of yeast expression plasmids like YEp6, YEp13, YEp4 can be used as vectors. A gene of interest can be fused to any of the promoters in various yeast vectors. The above-mentioned plasmids have been fully described in the literature (Botstein, et al., 1979, Gene, 8:17-24 (1979); Broach, et al., Gene, 8:121-133 (1979)).

Two procedures are used in transforming yeast cells. In one case, yeast cells are first converted into protoplasts using zymolyase, lyticase or glusulase, followed by addition of DNA and polyethylene glycol (PEG). The PEG-treated protoplasts are then regenerated in a 3% agar medium under selective conditions. Details of this procedure are given in the papers by J. D. Beggs, Nature (London), 275:104-109, (1978); and Hinnen, A., et al., Proc. Natl. Acad. Sci. USA, 75:1929-1933, (1978). The second procedure does not involve removal of the cell wall. Instead, the cells are treated with lithium chloride or acetate and PEG and put on selective plates (Ito, H., et al., J. Bact., 153:163-168 (1983)).

The protein can be isolated from yeast by lysing the cells and applying standard protein isolation techniques to the lysates. The monitoring of the purification process can be accomplished by using, for example, Western blot techniques or radioimmunoassays.

2. Expression in Insect Cells

The baculovirus expression vector utilizes the highly expressed and regulated Autographa californica nuclear polyhedrosis virus (AcMNPV) polyhedrin promoter modified for the insertion of foreign genes. Synthesis of polyhedrin protein results in the formation of occlusion bodies in the infected insect cell. The recombinant proteins expressed using this vector have been found in many cases to be antigenically, immunogenically and functionally similar to their natural counterparts. In addition, the baculovirus vector utilizes many of the protein modification, processing, and transport systems that occur in higher eukaryotic cells.

Briefly, the DNA sequence encoding, for example, the aging-associated protein is inserted into a transfer plasmid vector in the proper orientation downstream from the polyhedrin promoter, and flanked on both ends with baculovirus sequences. Cultured insect cell, commonly Spodoptera frugiperda, are transfected with a mixture of viral and plasmid DNAs. The virus that develop, some of which are recombinant virus that result from homologous recombination between the two DNAs, are plated at 100-1000 plaques per plate. The plaques containing recombinant virus can be identified visually because of their ability to form occlusion bodies or by DNA hybridization. The recombinant virus is isolated by plague purification. The resulting recombinant virus, capable of expressing, for example, an aging-associated protein, is self propagating in that no helper virus is required for maintenance or replication. After infecting an insect culture with recombinant virus, one can expect to find recombinant protein within 48-72 hours. The infection is essentially lytic within 4-5 days.

There are a variety of transfer vectors into which the nucleotides of the invention can be inserted. For a summary of transfer vectors, see, Luckow, V. A. and M. D. Summers, Bio/Technology, 6:47-55 (1988). Preferred is the transfer vector pAcUW21 described by Bishop, D. H. L. in Seminars in Virology, 3:253-264 (1992).

3. Expression in Recombinant Vaccinia Virus-Infected Cells

The gene encoding, for example, an aging-associated protein is inserted into a plasmid designed for producing recombinant vaccinia, such as pGS62, Langford, C. L., et al., Mol. Cell. Biol. 6:3191-3199, (1986). This plasmid consists of a cloning site for insertion of foreign genes, the P7.5 promoter of vaccinia to direct synthesis of the inserted gene, and the vaccinia TK gene flanking both ends of the foreign gene.

When the plasmid containing the desired nucleotide sequence is constructed, the gene can be transferred to vaccinia virus by homologous recombination in the infected cell. To achieve this, suitable recipient cells are transfected with the recombinant plasmid by standard calcium phosphate precipitation techniques into cells already infected with the desirable strain of vaccinia virus, such as Wyeth, Lister, W R or Copenhagen. Homologous recombination occurs between the TK gene in the virus and the flanking TK gene sequences in the plasmid. This results in a recombinant virus with the foreign gene inserted into the viral TK gene, thus rendering the TK gene inactive. Cells containing recombinant viruses are selected by adding medium containing 5-bromodeoxyuridine, which is lethal for cells expressing a TK gene.

Confirmation of production of recombinant virus can be achieved by DNA hybridization using cDNA encoding, for example, the aging-associated protein and by immunodetection techniques using antibodies specific for the expressed protein. Virus stocks may be prepared by infection of cells such as HeLA S3 spinner cells and harvesting of virus progeny.

4. Expression in Cell Cultures

The protein cDNA of the invention can be ligated to various expression vectors for use in transforming host cell cultures. The vectors typically contain gene sequences to initiate transcription and translation of the aging-associated gene. These sequences need to be compatible with the selected host cell. In addition, the vectors preferably contain a marker to provide a phenotypic trait for selection of transformed host cells such as dihydrofolate reductase or metallothionein. Additionally, a vector might contain a replicative origin.

Cells of mammalian origin are illustrative of cell cultures useful for the production of, for example, the aging-associated protein. Mammalian cell systems often will be in the form of monolayers of cells although mammalian cell suspensions may also be used. Illustrative examples of mammalian cell lines include VERO and HeLa cells, Chinese hamster ovary (CHO) cell lines, WI38, BHK, COS-7 or MDCK cell lines. NIH 3T3 or COS cells are preferred.

As indicated above, the vector, e.g., a plasmid, which is used to transform the host cell, preferably contains DNA sequences to initiate transcription and sequences to control the translation of the aging-associated protein gene sequence. These sequences are referred to as expression control sequences. Illustrative expression control sequences are obtained from the SV-40 promoter (Science, 222:524-527 (1983)), the CMV I.E. Promoter (Proc. Natl. Acad. Sci. 81:659-663 (1984)) or the metallothionein promoter (Nature 296:39-42 (1982)). The cloning vector containing the expression control sequences is cleaved using restriction enzymes and adjusted in size as necessary or desirable and ligated with sequences encoding the aging-associated protein by means well known in the art.

As with yeast, when higher animal host cells are employed, polyadenlyation or transcription terminator sequences from known mammalian genes need to be incorporated into the vector. An example of a terminator sequence is the polyadenlyation sequence from the bovine growth hormone gene. Sequences for accurate splicing of the transcript may also be included. An example of a splicing sequence is the VP1 intron from SV40 (Sprague, J. et al., J. Virol. 45: 773-781, (1983)).

Additionally, gene sequences to control replication in the host cell may be incorporated into the vector such as those found in bovine papilloma virus type-vectors. Saveria-Campo, M., “Bovine Papilloma virus DNA a Eukaryotic Cloning Vector” in DNA Cloning Vol.II a Practical Approach Ed. D. M. Glover, IRL Press, Arlington, Va. pp. 213-238, (1985).

The transformed cells are cultured by means well known in the art. For example, such means are published in Biochemical Methods in Cell Culture and Virology, Kuchler, R. J., Dowden, Hutchinson and Ross, Inc. (1977). The expressed protein is isolated from cells grown as suspensions or as monolayers. The latter are recovered by well known mechanical, chemical or enzymatic means.

Purification of the Proteins of the Invention

After expression, the proteins of the present invention can be purified to substantial purity by standard techniques, including selective precipitation with substances as ammonium sulfate; column chromatography; immunuopurification methods; and other methods known to those of skill in the art. See, for instance, R. Scopes, Protein Purification: Principles and Practice, Springer-Verlag: New York (1982), U.S. Pat. No. 4,673,641, Ausubel, and Sambrook, incorporated herein by reference.

A number of conventional procedures can be employed when recombinant protein is being purified. For example, proteins having established molecular adhesion properties can be reversible fused to the subject protein. With the appropriate ligand, the aging-associated protein, for example, can be selectively adsorbed to a purification column and then freed from the column in a relatively pure form. The fused protein is then removed by enzymatic activity. Finally, the aging-associated protein can be purified using immunoaffinity columns.

A. Purification of Proteins From Recombinant Bacteria

When recombinant proteins are expressed by the transformed bacteria in large amounts, typically after promoter induction, but expression can be constitutive, the proteins may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells, typically but not limited by, incubation in a buffer of about 100-150 g/mL lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Alternate methods of lysing bacteria are described in Ausubel and Sambrook and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties); the proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents which are capable of solubilizing aggregate-forming proteins, such as SDS (sodium dodecyl sulfate) and 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of immunologically and/or biologically active protein of interest. After solubilization, the protein can be separated from other bacterial proteins by standard separation techniques.

Alternatively, it is possible to purify protein from bacteria periplasm. Where protein is exported into the periplasm of the bacteria, the periplasmic fraction of the bacteria can be isolated by cold osmotic shock in addition to other methods known to skill in the art (see, Ausubel, supra).

To isolate recombinant proteins from the periplasm, the bacterial cells are centrifuiged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

B. Standard Protein Separation Techniques for Purifying Proteins

1. Solubility Fractionation

Often as an initial step, and if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic of proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

2. Size Differential Filtration

Based on a calculated molecular weight, this knowledge can be used to isolate the target protein of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

3. Column Chromatography

The target protein or protein of interest can also be separated from other proteins on the basis of their size, net surface charge, hydrophobicity and affinity for ligands. In addition, antibodies raised against proteins can be conjugated to column matrices and the proteins immunopurified. All of these methods are well known in the art.

It will be apparent to one of skill that chromatographic techniques can be performed at any scale and using equipment from many different manufacturers (e.g., Pharmacia Biotech).

Detection and Genomic Analysis of Aging-Associated Proteins.

The polynucleotides and polypeptides of the present invention can be employed as research reagents and materials for discovery of treatments and diagnostics to human disease. It will be readily apparent to those of skill in the art that methods for detecting nucleic acids associated with aging includes analysis of nucleic acids associated with particular phenotypes associated with aging, including, e.g. sensescence, cell proliferation, arrested cell growth, and/or nucleic acids associated with aging-assoicated diseases.

As should be apparent to those of skill, the invention is the identification of aging-associated genes and the discovery that multiple nucleic acids are associated with aging including such processes as senescence, cell proliferation, arrested cell growth and/or cell youthfulness. Accordingly, the present invention also includes methods for detecting the presence, alteration or absence of the aging-associated nucleic acid (e.g., DNA or RNA) in a physiological specimen in order to determine the aging status of cells in vitro, or ex vivo and their level of activity, e.g., proliferation state, risk that may be associated with a particular age-related genotype, or mutations that occur with aging. Although any tissue having cells bearing the genome of an individual, or cells expressing RNA associated with aging, can be used, the most convenient specimen will be blood samples or biopsies of suspect tissue. It is also possible and preferred in some circumstances to conduct assays on cells that are isolated under microscopic visualization. A particularly useful method is the microdissection technique described in PCT Published Application No. WO 95/23960. The cells isolated by microscopic visualization can be used in any of the assays described herein including both genomic and immunologic based assays.

This invention also provides methods of genotyping family members in which relatives are diagnosed with aging-related diseases. Conventional methods of genotyping are provided herein.

The invention provides methods for detecting whether a cell or tissue is aging. The methods typically comprise contacting RNA from the cell with a probe which comprises a polynucleotide sequence associated with aging; and determining whether the amount of the probe which hybridizes to the RNA is increased or decreased relative to the amount of the probe which hybridizes to RNA from a young cell. The assays are useful for detecting aging-associated diseases or diseases such as those associated with senescence, for example, Werner Syndrome and Progeria. One can also detect cell youthfulness or whether a cell is arrested at the G₀ stage of the cell cycle using the methods of the invention.

The probes are capable of binding to a target nucleic acid (e.g., a nucleic acid associated with aging). By assaying for the presence or absence of the probe, one can detect the presence or absence of the target nucleic acid in a sample. Preferably, non-hybridizing probe and target nucleic acids are removed (e.g., by washing) prior to detecting the presence of the probe.

A variety of methods of specific DNA and RNA measurement using nucleic acid hybridization techniques are known to those of skill in the art. See, Sambrook, supra. For example, one method for evaluating the presence or absence of the DNA in a sample involves a Southern transfer. Briefly, the digested genomic DNA is run on agarose slab gels in buffer and transferred to membranes. Hybridization is carried out using the probes discussed above. Visualization of the hybridized portions allows the qualitative determination of the presence, alteration or absence of an aging-associated gene.

Similarly, a Northern transfer may be used for the detection of aging-associated mRNA in samples of RNA from cells expressing the aging-associated proteins. In brief, the mRNA is isolated from a given cell sample using an acid guanidinium-phenol-chloroform extraction method. The mRNA is then electrophoresed to separate the mRNA species and the mRNA is transferred from the gel to a nitrocellulose membrane. As with the Southern blots, labeled probes are used to identify the presence or absence of the subject protein transcript. Alternatively, the amount of, for example, an aging-associated mRNA, mRNA can be analyzed in the absence of electrophoretic separation.

The selection of a nucleic acid hybridization format is not critical. A variety of nucleic acid hybridization formats are known to those skilled in the art. For example, common formats include sandwich assays and competition or displacement assays. Hybridization techniques are generally described in “Nucleic Acid Hybridization, A Practical Approach,” Ed. Hames, B. D. and Higgins, S. J., IRL Press, 1985; Gall and Pardue (1969), Proc. Natl. Acad. Sci., U.S.A., 63:378-383; and John, Burnsteil and Jones (1969) Nature, 223:582-587.

For example, sandwich assays are commercially useful hybridization assays for detecting or isolating nucleic acids. Such assays utilize a “capture” nucleic acid covalently immobilized to a solid support and labeled “signal” nucleic acid in solution. The clinical sample will provide the target nucleic acid. The “capture” nucleic acid and “signal” nucleic acid probe hybridize with the target nucleic acid to form a “sandwich” hybridization complex. To be effective, the signal nucleic acid cannot hybridize with the capture nucleic acid.

Detection of a hybridization complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal. The binding of the signal generation complex is also readily amenable to accelerations by exposure to ultrasonic energy.

The label may also allow indirect detection of the hybridization complex. For example, where the label is a hapten or antigen, the sample can be detected by using antibodies. In these systems, a signal is generated by attaching fluorescent or enzyme molecules to the antibodies or in some cases, by attachment to a radioactive label (see, e.g., Tijssen, P., “Practice and Theory of Enzyme Immunoassays,” Laboratory Techniques in Bio-chemistry and Molecular Biology, Burdon, R. H., van Knippenberg, P. H., Eds., Elsevier (1985), pp. 9-20).

The probes are typically labeled directly, as with isotopes, chromophores, lumiphores, chromogens, or indirectly labeled such as with biotin to which a streptavidin complex may later bind. Thus, the detectable labels used in the assays of the present invention can be primary labels (where the label comprises an element that is detected directly or that produces a directly detectable element) or secondary labels (where the detected label binds to a primary label, e.g., as is common in immunological labeling). Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labeled probes or the like.

Other labels include ligands which bind to labeled antibodies, fluorophores, chemiluminescent agents, enzymes, and antibodies which can serve as specific binding pair members for a labeled ligand. An introduction to labels, labeling procedures and detection of labels is found in Polak and Van Noorden (1997) Introduction to Immunocytochemistry, 2nd ed., Springer Verlag, New York, and in Haugland (1996) Handbook of Fluorescent Probes and Research Chemicals, a combined handbook and catalogue Published by Molecular Probes, Inc., Eugene, Oreg. Primary and secondary labels can include undetected elements as well as detected elements. Useful primary and secondary labels in the present invention can include spectral labels such as fluorescent dyes (e.g., fluorescein and derivatives such as fluorescein isothiocyanate (FITC) and Oregon Green™, rhodamine and derivatives (e.g., Texas red, tetrarhodimine isothiocynate (TRITC), etc.), digoxigenin, biotin, phycoerythrin, AMCA, CyDyes, and the like), radiolabels (e.g., ³H, ¹²⁵I, ³⁵S, ¹⁴C, ³²P, ³³P, etc.), enzymes (e.g., horse radish peroxidase, alkaline phosphatase etc.), spectral calorimetric labels such as colloidal gold or colored glass or plastic (e.g., polystyrene, polypropylene, latex, etc.) beads. The label may be coupled directly or indirectly to a component of the detection assay (e.g., the probe) according to methods well known in the art. As indicated above, a wide variety of labels may be used, with the choice of label depending on sensitivity required, ease of conjugation with the compound, stability requirements, available instrumentation, and disposal provisions.

Preferred labels include those that use: 1) chemiluminescence (using horseradish peroxidase and/or alkaline phosphatase with substrates that produce photons as breakdown products as described above) with kits being available, e.g., from Molecular Probes, Amersham, Boehringer-Mannheim, and Life Technologies/Gibco BRL; 2) color production (using both horseradish peroxidase and/or alkaline phosphatase with substrates that produce a colored precipitate [kits available from Life Technologies/Gibco BRL, and Boehringer-Mannheim]); 3) hemifluorescence using, e.g., alkaline phosphatase and the substrate AttoPhos [Amersham] or other substrates that produce fluorescent products, 4) fluorescence (e.g., using Cy-5 [Amersham]), fluorescein, and other fluorescent tags]; and 5) radioactivity. Other methods for labeling and detection will be readily apparent to one skilled in the art.

Preferred enzymes that can be conjugated to detection reagents of the invention include, e.g., β-galactosidase, luciferase, horse radish peroxidase, and alkaline phosphatase. The chemiluminescent substrate for luciferase is luciferin. One embodiment of a chemiluminescent substrate for β-galactosidase is 4-methylumbelliferyl-β-D-galactoside. Embodiments of alkaline phosphatase substrates include p-nitrophenyl phosphate (pNPP), which is detected with a spectrophotometer; 5-bromo-4-chloro-3-indolyl phosphate/nitro blue tetrazolium (BCIP/NBT) and fast red/napthol AS-TR phosphate, which are detected visually; and 4-methoxy-4-(3-phosphonophenyl) spiro[1,2-dioxetane-3,2′-adamantane], which is detected with a luminometer. Embodiments of horse radish peroxidase substrates include 2,2′azino-bis(3-ethylbenzthiazoline-6 sulfonic acid) (ABTS), 5-aminosalicylic acid (5AS), o-dianisidine, and o-phenylenediamine (OPD), which are detected with a spectrophotometer; and 3,3,5,5′-tetramethylbenzidine (TMB), 3,3′diaminobenzidine (DAB), 3-amino-9-ethylcarbazole (AEC), and 4-chloro-1-naphthol (4C1N), which are detected visually. Other suitable substrates are known to those skilled in the art. The enzyme-substrate reaction and product detection are performed according to standard procedures known to those skilled in the art and kits for performing enzyme immunoassays are available as described above.

In general, a detector which monitors a particular probe or probe combination is used to detect the detection reagent label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill. Commonly, an optical image of a substrate comprising bound labeling moieties is digitized for subsequent computer analysis.

Most typically, the amount of an aging-associated RNA is measured by quantitating the amount of label fixed to the solid support by binding of the detection reagent. Typically, presence of a modulator during incubation will increase or decrease the amount of label fixed to the solid support relative to a control incubation which does not comprise the modulator, or as compared to a baseline established for a particular reaction type. Means of detecting and quantitating labels are well known to those of skill in the art. Thus, for example, where the label is a radioactive label, means for detection include a scintillation counter or photographic film as in autoradiography. Where the label is optically detectable, typical detectors include microscopes, cameras, phototubes and photodiodes and many other detection systems which are widely available.

In preferred embodiments, the target nucleic acid or the probe is immobilized on a solid support. Solid supports suitable for use in the assays of the invention are known to those of skill in the art. As used herein, a solid support is a matrix of material in a substantially fixed arrangement. Exemplar solid supports include glasses, plastics, polymers, metals, metalloids, ceramics, organics, etc. Solid supports can be flat or planar, or can have substantially different conformations. For example, the substrate can exist as particles, beads, strands, precipitates, gels, sheets, tubing, spheres, containers, capillaries, pads, slices, films, plates, dipsticks, slides, etc. Magnetic beads or particles, such as magnetic latex beads and iron oxide particles, are examples of solid substrates that can be used in the methods of the invention. Magnetic particles are described in, for example, U.S. Pat. No. 4,672,040, and are commercially available from, for example, PerSeptive Biosystems, Inc. (Framingham Mass.), Ciba Coming (Medfield Mass.), Bangs Laboratories (Carmel Ind.), and BioQuest, Inc. (Atkinson N.H.). The substrate is chosen to maximize signal to noise ratios, primarily to minimize background binding, for ease of washing and cost.

A variety of automated solid-phase assay techniques are also appropriate. For instance, very large scale immobilized polymer arrays (VLSIPS™), available from Affymetrix, Inc. in Santa Clara, Calif. can be used to detect changes in expression levels of a plurality of aging-associated nucleic acids simultaneously. See, Tijssen, supra., Fodor et al. (1991) Science, 251: 767-777; Sheldon et al. (1993) Clinical Chemistry 39(4): 718-719, and Kozal et al. (1996) Nature Medicine 2(7): 753-759. Thus, in one embodiment, the invention provides methods of detecting aging-associated changes in expression levels of nucleic acids, in which nucleic acids (e.g., RNA from a cell culture), are hybridized to an array of nucleic acids that are known to be associated with aging. For example, in the assay described, supra, oligonucleotides which hybridize to a plurality of aging-associated nucleic acids are optionally synthesized on a DNA chip (such chips are available from Affymetrix) and the RNA from a biological sample, such as a cell culture, is hybridized to the chip for simultaneous analysis of multiple aging-related nucleic acids. The aging-associated nucleic acids that are present in the sample which is assayed are detected at specific positions on the chip.

Detection can be accomplished, for example, by using a labeled detection moiety that binds specifically to duplex nucleic acids (e.g., an antibody that is specific for RNA-DNA duplexes). One preferred example uses an antibody that recognizes DNA-RNA heteroduplexes in which the antibody is linked to an enzyme (typically by recombinant or covalent chemical bonding). The antibody is detected when the enzyme reacts with its substrate, producing a detectable product. Coutlee et al. (1989) Analytical Biochemistry 181:153-162; Bogulavski et al. (1986) J. Immunol. Methods 89:123-130; Prooijen-Knegt (1982) Exp. Cell Res. 141:397-407; Rudkin (1976) Nature 265:472-473, Stollar (1970) PNAS 65:993-1000; Ballard (1982) Mol. Immunol. 19:793-799; Pisetsky and Caster (1982) Mol. Immunol. 19:645-650; Viscidi et al. (1988) J. Clin. Microbial. 41:199-209, and Kiney et al. (1989) J. Clin. Microbiol. 27:6-12 describe antibodies to RNA duplexes, including homo and heteroduplexes. Kits comprising antibodies specific for DNA:RNA hybrids are available, e.g., from Digene Diagnostics, Inc. (Beltsville, Md.).

In addition to available antibodies, one of skill can easily make antibodies specific for nucleic acid duplexes using existing techniques, or modify those antibodies which are commercially or publicly available. In addition to the art referenced above, general methods of producing polyclonal and monoclonal antibodies are known to those of skill in the art. See, e.g., Paul (ed) (1993) Fundamental Immunology, Third Edition Raven Press, Ltd., New York Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Goding (1986) Monoclonal Antibodies: Principles and Practice (2d ed.) Academic Press, New York, N.Y.; and Kohler and Milstein (1975) Nature 256: 495-497. Other suitable techniques for antibody preparation include selection of libraries of recombinant antibodies in phage or similar vectors. See, Huse et al. (1989) Science 246: 1275-1281; and Ward et al. (1989) Nature 341: 544-546. Specific monoclonal and polyclonal antibodies and antisera will usually bind with a K_{D} of at least about 0.1 μM, preferably at least about 0.01 μM or better, and most typically and preferably, 0.001 μM or better.

The nucleic acids used in this invention can be either positive or negative probes. Positive probes bind to their targets and the presence of duplex formation is evidence of the presence of the target. Negative probes fail to bind to the suspect target and the absence of duplex formation is evidence of the presence of the target. For example, the use of a wild type specific nucleic acid probe or PCR primers may act as a negative probe in an assay sample where only the nucleotide sequence of interest is present.

The sensitivity of the hybridization assays may be enhanced through use of a nucleic acid amplification system which multiplies the target nucleic acid being detected. Examples of such systems include the polymerase chain reaction (PCR) system and the ligase chain reaction (LCR) system. Other methods recently described in the art are the nucleic acid sequence based amplification (NASBA [symbol] , Cangene, Mississauga, Ontario) and Q Beta Replicase systems. These systems can be used to directly identify mutants where the PCR or LCR primers are designed to be extended or ligated only when a select sequence is present. Alternatively, the select sequences can be generally amplified using, for example, nonspecific PCR primers and the amplified target region later probed for a specific sequence indicative of a mutation.

In one embodiment, allelic specific amplification can be used. In the case of PCR, the amplification primers are designed to bind to a portion of, for example, the aging-associated gene, but the terminal base at the 3′ end is used to discriminate between the mutant and wild-type forms of the age-associated target protein gene. If the terminal base matches the point mutation or the wild-type, polymerase dependent three prime extension can proceed and an amplification product is detected. This method for detecting point mutations or polymorphisms is described in detail by Sommer, S. S., et al., in Mayo Clin. Proc. 64:1361-1372,(1989), incorporated herein by reference. By using appropriate controls, one can develop a kit having both positive and negative amplification products. The products can be detected using specific probes or by simply detecting their presence or absence. A variation of the PCR method uses LCR where the point of discrimination, i.e, either the point mutation or the wild-type bases fall between the LCR oligonucleotides. The ligation of the oligonucleotides becomes the means for discriminating between the mutant and wild-type forms of the target protein gene.

An alternative means for determining the level of expression of the nucleic acids of the present invention is in situ hybridization. In situ hybridization assays are well known and are generally described in Angerer, et al., Methods Enzymol., 152:649-660 (1987). In an in situ hybridization assay cells, preferentially bovine lymphocytes are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled with radioisotopes or fluorescent reporters.

Immunological Detection of Target Protein

In addition to the detection of the target protein gene expression using nucleic acid hybridization technology, one can also use immunoassays to detect target protein. Immunoassays can be used to qualitatively or quantitatively analyze the proteins of interest. A general overview of the applicable technology can be found in Harlow and Lane, Antibodies: A Laboratory Manual, Cold Spring Harbor Pubs., N.Y. (1988), incorporated herein by reference. Although the following discussion is directed to methods for detecting target aging-associated proteins, similar methods can be used to detect proteins associated with other parameters that may be associated with aging in specific cells or tissues such as cell proliferation, cell youthfulness, arrested cell growth and/or target proteins associated with aging-related diseases (e.g., Werner Syndrome, Progeria, etc.) or diseases typically associated with aging such as heart disease.

A. Antibodies to Target Proteins

Methods of producing polyclonal and monoclonal antibodies that react specifically with a protein of interest are known to those of skill in the art. See, e.g., Coligan (1991), CURRENT PROTOCOLS IMMUNOLOGY, Wiley/Greene, NY; and Harlow and Lane; Stites et al. (eds.) BASIC AND CLINICAL IMMUNOLOGY (4th ed.) Lange Medical Publications, Los Altos, Calif., and references cited therein; Goding (1986), MONOCLONAL ANTIBODIES: PRINCIPLES AND PRACTICE (2d ed.) Academic Press, New York, N.Y.; and Kohler and Milstein (1975), Nature, 256:495-497. Such techniques include antibody preparation by selection of antibodies from libraries of recombinant antibodies in phage or similar vectors. See, Huse et al. (1989), Science, 246:1275-1281; and Ward et al. (1989), Nature, 341:544-546. For example, in order to produce antisera for use in an immunoassay, the proteins of interest or an antigenic fragment thereof, is isolated as described herein. For example, recombinant protein is produced in a transformed cell line. An inbred strain of mice or rabbits is immunized with the protein using a standard adjuvant, such as Freund's adjuvant, and a standard immunization protocol. Alternatively, a synthetic peptide derived from the sequences disclosed herein and conjugated to a carrier protein can be used as an immunogen.

Polyclonal sera are collected and titered against the immunogen protein in an immunoassay, for example, a solid phase immunoassay with the immunogen immobilized on a solid support. Polyclonal antisera with a titer of 10⁴ or greater are selected and tested for their cross reactivity against other proteins or even other homologous proteins from other organisms, using a competitive binding immunoassay. Specific monoclonal and polyclonal antibodies and antisera will usually bind with a K_{D} of at least about 0.1 mM, more usually at least about 1 μM, preferably at least about 0.1 μM or better, and most preferably, 0.01 μM or better.

A number of proteins of the invention comprising immunogens may be used to produce antibodies specifically or selectively reactive with the proteins of interest. Recombinant protein is the preferred immunogen for the production of monoclonal or polyclonal antibodies. Naturally occurring protein may also be used either in pure or impure form. Synthetic peptides made using the protein sequences described herein may also used as an immunogen for the production of antibodies to the protein. Recombinant protein can be expressed in eukaryotic or prokaryotic cells as described above, and purified as generally described above. The product is then injected into an animal capable of producing antibodies. Either monoclonal or polyclonal antibodies may be generated, for subsequent use in immunoassays to measure the protein.

Methods of production of polyclonal antibodies are known to those of skill in the art. In brief, an immunogen, preferably a purified protein, is mixed with an adjuvant and animals are immunized. The animal's immune response to the immunogen preparation is monitored by taking test bleeds and determining the titer of reactivity to the aging-associated target protein. When appropriately high titers of antibody to the immunogen are obtained, blood is collected from the animal and antisera are prepared. Further fractionation of the antisera to enrich for antibodies reactive to the protein can be done if desired (see, Harlow and Lane, supra).

Monoclonal antibodies may be obtained by various techniques familiar to those skilled in the art. Briefly, spleen cells from an animal immunized with a desired antigen are immortalized, commonly by fusion with a myeloma cell (See, Kohler and Milstein, Eur. J. Immunol. 6:511-519 (1976), incorporated herein by reference). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al. (1989) Science 246:1275-1281.

Once target protein specific antibodies are available, the protein can be measured by a variety of immunoassay methods with qualitative and quantitative results available to the clinician. For a review of immunological and immunoassay procedures in general (see, Basic and Clinical Immunology 7th Edition (D. Stites and A. Terr ed.) 1991). Moreover, the immunoassays of the present invention can be performed in any of several configurations, which are reviewed extensively in Enzyme Immunoassay, E. T. Maggio, ed., CRC Press, Boca Raton, Fla. (1980); “Practice and Theory of Enzyme Immunoassays,” Tijssen; and, Harlow and Lane, each of which is incorporated herein by reference.

Immunoassays to measure target proteins in a human sample may use a polyclonal antiserum which was raised to the protein partially encoded by a sequence described herein or a fragment thereof. This antiserum is selected to have low crossreactivity against other proteins and any such crossreactivity is removed by immunoabsorption prior to use in the immunoassay.

In order to produce antisera for use in an immunoassay, an aging-associated target protein or a fragment thereof, for example, is isolated as described herein. For example, recombinant protein is produced in a transformed cell line. An inbred strain of mice, such as Balb/c, is immunized with the protein or a peptide using a standard adjuvant, such as Freund's adjuvant, and a standard mouse immunization protocol. Alternatively, a synthetic peptide derived from the sequences disclosed herein and conjugated to a carrier protein can be used an immunogen. Polyclonal sera are collected and titered against the immunogen protein in an immunoassay, for example, a solid phase immunoassay with the immunogen immobilized on a solid support. Polyclonal antisera with a titer of 10⁴ or greater are selected and tested for their cross reactivity against other proteins, using a competitive binding immunoassay such as the one described in Harlow and Lane, supra, at pages 570-573 and below.

B. Immunological Binding Assays

In a preferred embodiment, a protein of interest is detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology Volume 37: Antibodies in Cell Biology, Asai, ed. Academic Press, Inc. New York (1993); Basic and Clinical Immunology 7th Edition, Stites & Terr, eds. (1991). Immunological binding assays (or immunoassays) typically utilize a “capture agent” to specifically bind to and often immobilize the analyte (in this case the aging-associated target protein or antigenic subsequence thereof). The capture agent is a moiety that specifically binds to the analyte. In a preferred embodiment, the capture agent is an antibody that specifically binds, for example, the aging-associated target protein. The antibody (e.g., anti-target protein) may be produced by any of a number of means well known to those of skill in the art and as described above.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled target aging-associated protein polypeptide or a labeled anti-target protein antibody. Alternatively, the labeling agent may be a third moiety, such as another antibody, that specifically binds to the antibody/protein complex.

In a preferred embodiment, the labeling agent is a second antibody bearing a label. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

Other proteins capable of specifically binding immunoglobulin constant regions, such as protein A or protein G, can also be used as the label agent. These proteins are normal constituents of the cell walls of streptococcal bacteria. They exhibit a strong non-immunogenic reactivity with immunoglobulin constant regions from a variety of species (see, generally, Kronval, et al. (1973) J. Immunol., 111: 1401-1406, and Akerstrom, et al. (1985) J. Immunol., 135: 2589-2542).

Throughout the assays, incubation and/or washing steps may be required after each combination of reagents. Incubation steps can vary from about 5 seconds to several hours, preferably from about 5 minutes to about 24 hours. However, the incubation time will depend upon the assay format, analyte, volume of solution, concentrations, and the like. Usually, the assays will be carried out at ambient temperature, although they can be conducted over a range of temperatures, such as 10° C. to 40° C.

1. Non-Competitive Assay Formats

Immunoassays for detecting proteins of interest from tissue samples may be either competitive or noncompetitive. Noncompetitive immunoassays are assays in which the amount of captured analyte (in this case the target aging-associated protein) is directly measured. In one preferred “sandwich” assay, for example, the capture agent (e.g., anti-target protein antibodies) can be bound directly to a solid substrate where they are immobilized. These immobilized antibodies then capture the target protein present in the test sample. The target protein thus immobilized is then bound by a labeling agent, such as a second antibody bearing a label. Alternatively, the second antibody may lack a label, but it may, in turn, be bound by a labeled third antibody specific to antibodies of the species from which the second antibody is derived. The second can be modified with a detectable moiety, such as biotin, to which a third labeled molecule can specifically bind, such as enzyme-labeled streptavidin.

2. Competitive Assay Formats

In competitive assays, the amount of target protein (analyte) present in the sample is measured indirectly by measuring the amount of an added (exogenous) analyte (i.e., the target protein) displaced (or competed away) from a capture agent (anti-target protein antibody) by the analyte present in the sample. In one competitive assay, a known amount of, in this case, the target protein is added to the sample and the sample is then contacted with a capture agent, in this case an antibody that specifically binds to the target protein. The amount of target protein bound to the antibody is inversely proportional to the concentration of target protein present in the sample. In a particularly preferred embodiment, the antibody is immobilized on a solid substrate. The amount of the target protein bound to the antibody may be determined either by measuring the amount of target protein present in a target protein/antibody complex or, alternatively, by measuring the amount of remaining uncomplexed protein. The amount of target protein may be detected by providing a labeled target protein molecule.

A hapten inhibition assay is another preferred competitive assay. In this assay, a known analyte, in this case the target protein, is immobilized on a solid substrate. A known amount of anti-target protein antibody is added to the sample, and the sample is then contacted with the immobilized target. In this case, the amount of anti-target protein antibody bound to the immobilized target protein is inversely proportional to the amount of target protein present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

Immunoassays in the competitive binding format can be used for crossreactivity determinations. For example, the protein encoded by the sequences described herein can be immobilized to a solid support. Proteins are added to the assay which compete with the binding of the antisera to the immobilized antigen. The ability of the above proteins to compete with the binding of the antisera to the immobilized protein is compared to the protein encoded by any of the sequences described herein. The percent crossreactivity for the above proteins is calculated, using standard calculations. Those antisera with less than 10% crossreactivity with each of the proteins listed above are selected and pooled. The cross-reacting antibodies are optionally removed from the pooled antisera by immunoabsorption with the considered proteins, e.g., distantly related homologues.

The immunoabsorbed and pooled antisera are then used in a competitive binding immunoassay as described above to compare a second protein, thought to be perhaps the protein of this invention, to the immunogen protein. In order to make this comparison, the two proteins are each assayed at a wide range of concentrations and the amount of each protein required to inhibit 50% of the binding of the antisera to the immobilized protein is determined. If the amount of the second protein required is less than 10 times the amount of the protein partially encoded by a sequence herein that is required, then the second protein is said to specifically bind to an antibody generated to an immunogen consisting of the target protein.

3. Other Assay Formats

In a particularly preferred embodiment, Western blot (immunoblot) analysis is used to detect and quantify the presence of target protein in the sample. The technique generally comprises separating sample proteins by gel electrophoresis on the basis of molecular weight, transferring the separated proteins to a suitable solid support (such as a nitrocellulose filter, a nylon filter, or derivatized nylon filter) and incubating the sample with the antibodies that specifically bind the target protein. For example, the anti-target protein antibodies specifically bind to the target protein on the solid support. These antibodies may be directly labeled or alternatively may be subsequently detected using labeled antibodies (e.g., labeled sheep anti-mouse antibodies) that specifically bind to the anti-target protein antibodies.

Other assay formats include liposome immunoassays (LIA), which use liposomes designed to bind specific molecules (e.g., antibodies) and release encapsulated reagents or markers. The released chemicals are then detected according to standard techniques (see, Monroe et al. (1986) Amer. Clin. Prod. Rev. 5:34-41).

4. Reduction of Non-Specific Binding

One of skill in the art will appreciate that it is often desirable to use non-specific binding in immunoassays. Particularly, where the assay involves an antigen or antibody immobilized on a solid substrate it is desirable to minimize the amount of non-specific binding to the substrate. Means of using such non-specific binding are well known to those of skill in the art. Typically, this involves coating the substrate with a proteinaceous composition. In particular, protein compositions, such as bovine serum albumin (BSA), nonfat powdered milk and gelatin, are widely used with powdered milk being most preferred.

5. Labels

The particular label or detectable group used in the assay is not a critical aspect of the invention, so long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., Dynabeads™), fluorescent dyes (e.g., fluorescein isothiocyanate, Texas red, rhodamine, and the like), radiolabels (e.g., ³H, ¹²⁵, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Thyroxine, and cortisol can be used in conjunction with the labeled, naturally occurring anti-ligands. Alternatively, any haptenic or antigenic compound can be used in combination with an antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems which may be used, see, U.S. Pat. No. 4,391,904).

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

Screening for Modulators of Aging-Associated Changes in Expression

The invention also provides methods of identifying compounds that modulate the aging process, e.g., by modulating the expression or activity of one or more aging-associated protein or nucleic acid sequences. For example, the methods can identify compounds that increase or decrease the expression level of genes associated with aging and that modulate processes often changed with aging such as senescence, loss of proliferative capacity, oxidation of lipids and proteins and other cellular and physiological aging-related phenotypes. Although the following discussion is directed to methods for screening for modulators of aging-associated changes in gene expression, similar methods can be used to screen for modulators of aging-associated phenotypic features such as cell proliferation, or modulators of activity of proteins encoded by aging-associated genes.

Compounds that are identified as modulators of aging-associated changes in expression using the methods of the invention find use both in vitro and in vivo. For example, one can treat cell cultures with the modulators in experiments designed to determine the mechanisms by which aging is regulated. Compounds that decrease or delay altered aging-associated gene expression or aging-associated protein activity are useful for extending the useful life of cell cultures, for example, those that are used for production of biological products such as recombinant proteins. In vivo uses of compounds that delay aging-associated phenotypic changes include, for example, delaying the aging process and treating diseases often associated with aging, such as heart disease or cancer. Conversely, compounds that accelerate or increase aging-associated changes can be useful, for example, as anticancer agents, as cancer is often associated with a loss of a cell's ability to undergo phenotypic changes typically associated with a mature cell population.

The methods typically involve culturing a cell in the presence of a potential modulator to form a first cell culture. RNA from the first cell culture is contacted with a probe which comprises a polynucleotide sequence associated with aging. The amount of the probe which hybridizes to the RNA from the first cell culture is determined. Typically, one determines whether the amount of probe which hybridizes to the RNA is increased or decrease relative to the amount of the probe which hybridizes to RNA from a second cell culture grown in the absence of the modulator.

It may be further determined whether the modulator-induced increase or decrease in RNA levels of the target sequence is correlated with an age-associated change in cellular phenotype. For example, a fibroblast cell population that is treated with a modulator which induces decreased expression of a gene that is normally upregulated with aging, or a fibroblast cell that is treated with a modulator which induces increased expression of a gene that is normally downregulated with aging, may be further tested for regained proliferative potential, which is reflective of a “younger” phenotype. Frequently, a young phenotype is the phenotype observed in cells or tissues that are obtained from an individual of about 30 years or less in age, whereas an aged phenotype is the phenotype observed in cells or tissues that are obtained from an individual of about 65 years or less in age.

Essentially any chemical compound can be used as a potential modulator in the assays of the invention, although most often compounds can be dissolved in aqueous or organic (for example, DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial library containing a large number of potential therapeutic compounds (potential modulator compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (PCT Publication No. WO 91/19735), encoded peptides (PCT Publication WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with β-D-glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MNS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

As noted, the invention provides in vitro assays for identifying, in a high throughput format, compounds that can modulate changes in expression and activity of aging-associated nucleic acids and proteins. Control reactions that measure the change in expression or aging-associated phenotype of the cell in a reaction that does not include a potential modulator are optional, as the assays are highly uniform. Such optional control reactions are appropriate and increase the reliability of the assay. Accordingly, in a preferred embodiment, the methods of the invention include such a control reaction. For each of the assay formats described, “no modulator” control reactions which do not include a modulator provide a background level of binding activity.

In some assays it will be desirable to have positive controls to ensure that the components of the assays are working properly. At least two types of positive controls are appropriate. First, a known activator of an aging-associated gene or protein can be incubated with one sample of the assay, and the resulting increase in signal resulting from an increased expression level of a gene associated with aging determined according to the methods herein. Second, a known inhibitor of an aging-associated gene or protein can be added, and the resulting decrease in signal similarly detected. It will be appreciated that modulators can also be combined with activators or inhibitors to find modulators which inhibit the increase or decrease that is otherwise caused by the presence of the known modulator of the aging-associated sequence or aging-associated phenotype.

In the high throughput assays of the invention, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (96) modulators. If 1536 well plates are used, then a single plate can easily assay from about 100- about 1500 different compounds. It is possible to assay many different plates per day; assay screens for up to about 6,000-20,000, and even up to about 100,000 different compounds is possible using the integrated systems of the invention.

Compositions, Kits and Integrated Systems

The invention provides compositions, kits and integrated systems for practicing the assays described herein. The following discussion is directed to kits for carrying out assays using nucleic acids (or proteins, antibodies, etc.) exhibiting altered expression or activity with aging. For instance, an assay composition having a nucleic acid that undergoes an age-associating change in expression, and a labelling reagent is provided by the present invention. In preferred embodiments, a plurality of, for example, aging-associated nucleic acids are provided in the assay compositions. The invention also provides assay compositions for use in solid phase assays; such compositions can include, for example, one or more aging-associated nucleic acids immobilized on a solid support, and a labelling reagent. In each case, the assay compositions can also include additional reagents that are desirable for hybridization. Modulators of expression of aging-related nucleic acids or modulators of activity of aging-related proteins can also be included in the assay compositions.

The invention also provides kits for carrying out the assays of the invention. The kits typically include a probe which comprises a polynucleotide sequence associated with aging; and a label for detecting the presence of the probe. Preferably, the kits will include a plurality of polynucleotide sequences associated with aging. Kits can include any of the compositions noted above, and optionally further include additional components such as instructions to practice a high-throughput method of assaying for an effect on aging and expression of aging-related genes, one or more containers or compartments (e.g., to hold the probe, labels, or the like), a control modulator of aging, a robotic armature for mixing kit components or the like.

The invention also provides integrated systems for high-throughput screening of potential modulators for an effect on cell aging (e.g., changes in expression patterns associated with aging). The systems typically include a robotic armature which transfers fluid from a source to a destination, a controller which controls the robotic armature, a label detector, a data storage unit which records label detection, and an assay component such as a microtiter dish comprising a well having a reaction mixture or a substrate comprising a fixed nucleic acid or immobilization moiety.

A number of robotic fluid transfer systems are available, or can easily be made from existing components. For example, a Zymate XP (Zymark Corporation; Hopkinton, Mass.) automated robot using a Microlab 2200 (Hamilton; Reno, Nev.) pipetting station can be used to transfer parallel samples to 96 well microtiter plates to set up several parallel simultaneous STAT binding assays.

Optical images viewed (and, optionally, recorded) by a camera or other recording device (e.g., a photodiode and data storage device) are optionally further processed in any of the embodiments herein, e.g., by digitizing the image and storing and analyzing the image on a computer. A variety of commercially available peripheral equipment and software is available for digitizing, storing and analyzing a digitized video or digitized optical image, e.g., using PC (Intel x86 or Pentium chip-compatible DOS®, OS2® WINDOWS®, WINDOWS NT® or WINDOWS95® based computers), MACINTOSH®, or UNIX® based (e.g., SUN® work station) computers.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen (e.g., individual hybridization sites on an array of biological polymers) are sampled to obtain light intensity readings for each position. Multiple pixels are processed in parallel to increase speed. The apparatus and methods of the invention are easily used for viewing any sample, e.g., by fluorescent or dark field microscopic techniques.

Gene Therapy Applications

A variety of human diseases can be treated by therapeutic approaches that involve stably introducing a gene into a human cell such that the gene is transcribed and the gene product is produced in the cell. Diseases amenable to treatment by this approach include inherited diseases, including those in which the defect is in a single gene. Gene therapy is also useful for treatment of acquired diseases and other conditions such as diseases typically associated with aging, e.g., heart disease and cancer. For discussions on the application of gene therapy towards the treatment of genetic as well as acquired diseases. See, Miller, A. D. (1992) Nature 357:455-460, and Mulligan, R. C. (1993) Science 260:926-932, both of which are incorporated herein by reference.

Nucleic acids that can be administered to prevent or reduce aging-associated changes in gene expression include aging-associated genes that are underexpressed with aging, in order to increase the expression level of the aging-associated gene, or alternatively, inhibitory nucliec acids that target genes that exhibit increased expression with aging. Additionally, gene therapy can be achieved by administration of nucleic acids that serve as activators or inhibitors of expression of the aging-associated nucleic acid identified herein.

A. Vectors for Gene Delivery

For delivery to a cell or organism, the nucleic acids of the invention can be incorporated into a vector. Examples of vectors used for such purposes include expression plasmids capable of directing the expression of the nucleic acids in the target cell. In other instances, the vector is a viral vector system wherein the nucleic acids are incorporated into a viral genome that is capable of transfecting the target cell. In a preferred embodiment, the nucleic acids can be operably linked to expression and control sequences that can direct expression of the gene in the desired target host cells. Thus, one can achieve expression of the nucleic acid under appropriate conditions in the target cell.

B. Gene Delivery Systems

Viral vector systems useful in the expression of the nucleic acids include, for example, naturally occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus, minute virus of mice (MVM), HIV, sindbis virus, and retroviruses (including but not limited to Rous sarcoma virus), and MoMLV. Typically, genes of interest are inserted into such vectors to allow packaging of the gene construct, typically with accompanying viral DNA, followed by infection of a sensitive host cell and expression of the gene of interest.

As used herein, “gene delivery system” refers to any means for the delivery of a nucleic acid of the invention to a target cell. In some embodiments of the invention, nucleic acids are conjugated to a cell receptor ligand for facilitated uptake (e.g., invagination of coated pits and internalization of the endosome) through an appropriate linking moiety, such as a DNA linking moiety (Wu et al., J. Biol. Chem. 263:14621-14624 (1988); WO 92/06180). For example, nucleic acids can be linked through a polylysine moiety to asialooromucocid, which is a ligand for the asialoglycoprotein receptor of hepatocytes.

Similarly, viral envelopes used for packaging gene constructs that include the nucleic acids of the invention can be modified by the addition of receptor ligands or antibodies specific for a receptor to permit receptor-mediated endocytosis into specific cells (see, e.g., WO 93/20221, WO 93/14188, WO 94/06923). In some embodiments of the invention, the DNA constructs of the invention are linked to viral proteins, such as adenovirus particles, to facilitate endocytosis (Curiel et al., Proc. Natl. Acad. Sci. U.S.A. 88: 8850-8854 (1991)). In other embodiments, molecular conjugates of the instant invention can include microtubule inhibitors (WO/9406922); synthetic peptides mimicking influenza virus hemagglutinin (Plank et al., J. Biol. Chem. 269:12918-12924 (1994)); and nuclear localization signals such as SV40 T antigen (WO93/19768).

Retroviral vectors are also useful for introducing the nucleic acids of the invention into target cells or organisms. Retroviral vectors are produced by genetically manipulating retroviruses. Retroviruses are called RNA viruses because the viral genome is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild type retroviral genome and the proviral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site). See, Mulligan, R. C., In: Experimental Manipulation of Gene Expression, M. Inouye (ed), 155-173 (1983); Mann, R., et al., Cell, 33:153-159 (1983); Cone, R. D. and R. C. Mulligan, Proceedings of the National Academy of Sciences, U.S.A., 81:6349-6353 (1984).

The design of retroviral vectors is well known to those of ordinary skill in the art. See, e.g., Singer, M. and Berg, P., supra. In brief, if the sequences necessary for encapsidation (or packaging of retroviral RNA into infectious virions) are missing from the viral genome, the result is a cis acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including European Patent Application EPA 0 178 220, U.S. Pat. No. 4,405,712, Gilboa, Biotechniques 4:504-512 (1986), Mann, et al., Cell 33:153-159 (1983), Cone and Mulligan, Proc. Natl. Acad. Sci. USA 81:6349-6353 (1984), Eglitis, M. A, et al. (1988) Biotechniques 6:608-614, Miller, A. D. et al. (1989) Biotechniques 7:981-990, Miller, A. D.(1992) Nature, supra, Mulligan, R. C. (1993), supra, and Gould, B. et al., and International Publication No. WO 92/07943 entitled “Retroviral Vectors Useful in Gene Therapy”. The teachings of these patents and publications are incorporated herein by reference.

The retroviral vector particles are prepared by recombinantly inserting the desired nucleotide sequence into a retrovirus vector and packaging the vector with retroviral capsid proteins by use of a packaging cell line. The resultant retroviral vector particle is incapable of replication in the host cell and is capable of integrating into the host cell genome as a proviral sequence containing the desired nucleotide sequence. As a result, the patient is capable of producing an aging-associated protein or a protein that prevents aging-associated changes in expression and thus restore the cells to a young phenotype.

Packaging cell lines that are used to prepare the retroviral vector particles are typically recombinant mammalian tissue culture cell lines that produce the necessary viral structural proteins required for packaging, but which are incapable of producing infectious virions. The defective retroviral vectors that are used, on the other hand, lack the these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these-cells, the gag, pol, and env genes can be derived from the same or different retroviruses.

A number of packaging cell lines suitable for the present invention are also available in the prior art. Examples of these cell lines include Crip, GPE86, PA317 and PG13. See Miller et al., J. Virol. 65:2220-2224 (1991), which is incorporated herein by reference. Examples of other packaging cell lines are described in Cone, R. and Mulligan, R. C., Proceedings of the National Academy of Sciences, USA, 81:6349-6353 (1984) and in Danos, O. and R. C. Mulligan, Proceedings of the National Academy of Sciences, USA, 85: 6460-6464 (1988), Eglitis, M. A., et al. (1988), supra, and Miller, A. D., (1990), supra, also all incorporated herein by reference.

Packaging cell lines capable of producing retroviral vector particles with chimeric envelope proteins may be used. Alternatively, amphotropic or xenotropic envelope proteins, such as those produced by PA317 and GPX packaging cell lines may be used to package the retroviral vectors.

In some embodiments of the invention, an antisense nucleic acid is administered which hybridizes to a gene associated with aging, or a phenotype often associated with aging such as senescence, G₀, or the like, or to transcript thereof. The antisense nucleic acid can be provided as an antisense oligonucleotide (see, e.g., Murayama et al., Antisense Nucleic Acid Drug Dev. 7:109-114 (1997)). Genes encoding an antisense nucleic acid can also be provided; such genes can be introduced into cells by methods known to those of skill in the art. For example, one can introduce a gene that encodes an antisense nucleic acid in a viral vector, such as, for example, in hepatitis B virus (see, e.g., Ji et al., J. Viral Hepat. 4:167-173 (1997)); in adeno-associated virus (see, e.g., Xiao et al., Brain Res. 756:76-83 (1997)); or in other systems including, but not limited, to an HVJ (Sendai virus)-liposome gene delivery system (see, e.g., Kaneda et al., Ann. N.Y. Acad. Sci. 811:299-308 (1997)); a “peptide vector” (see, e.g., Vidal et al., CR Acad. Sci III 32:279-287 (1997)); as a gene in an episomal or plasmid vector (see, e.g., Cooper et al., Proc. Natl. Acad. Sci. U.S.A. 94:6450-6455 (1997), Yew et al. Hum Gene Ther. 8:575-584 (1997)); as a gene in a peptide-DNA aggregate (see, e.g., Niidome et al., J. Biol. Chem. 272:15307-15312 (1997)); as “naked DNA” (see, e.g., U.S. Pat. No. 5,580,859 and U.S. Pat. No. 5,589,466); in lipidic vector systems (see, e.g., Lee et al., Crit Rev Ther Drug Carrier Syst. 14:173-206 (1997)); polymer coated liposomes (Marin et al., U.S. Pat. No. 5,213,804, issued May 25, 1993; Woodle et al., U.S. Pat. No. 5,013,556, issued May 7, 1991); cationic liposomes (Epand et al., U.S. Pat. No. 5,283,185, issued Feb. 1, 1994; Jessee, J. A., U.S. Pat. No. 5,578,475, issued Nov. 26, 1996; Rose et al, U.S. Pat. No. 5,279,833, issued Jan. 18, 1994; Gebeyehu et al., U.S. Pat. No. 5,334,761, issued Aug. 2, 1994); gas filled microspheres (Unger et al., U.S. Pat. No. 5,542,935, issued Aug. 6, 1996), ligand-targeted encapsulated macromolecules (Low et al. U.S. Pat. No. 5,108,921, issued Apr. 28, 1992; Curiel et al., U.S. Pat. No. 5,521,291, issued May 28, 1996; Groman et al., U.S. Pat. No. 5,554,386, issued Sep. 10, 1996; Wu et al., U.S. Pat. No. 5,166,320, issued Nov. 24, 1992).

C. Pharmaceutical Formulations

When used for pharmaceutical purposes, the vectors used for gene therapy are formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al. (1966) Biochemistry 5:467.

The compositions can additionally include a stabilizer, enhancer or other pharmaceutically acceptable carriers or vehicles. A pharmaceutically acceptable carrier can contain a physiologically acceptable compound that acts, for example, to stabilize the nucleic acids of the invention and any associated vector. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. Examples of carriers, stabilizers or adjuvants can be found in Martin, Remington's Pharm. Sci., 15th Ed. (Mack Publ. Co., Easton, Pa. 1975), which is incorporated herein by reference.

D. Administration of Formulations

The formulations of the invention can be delivered to any tissue or organ using any delivery method known to the ordinarily skilled artisan for example. In some embodiments of the invention, the nucleic acids of the invention are formulated in mucosal, topical, and/or buccal formulations, particularly mucoadhesive gel and topical gel formulations. Exemplary permeation enhancing compositions, polymer matrices, and mucoadhesive gel preparations for transdermal delivery are disclosed in U.S. Pat. No. 5,346,701. In some embodiments of the invention, a therapeutic agent is formulated in ophthalmic formulations for administration to the eye.

E. Methods of Treatment

The gene therapy formulations of the invention are typically administered to a cell. The cell can be provided as part of a tissue, such as an epithelial membrane, or as an isolated cell, such as in tissue culture. The cell can be provided in vivo, ex vivo, or in vitro.

The formulations can be introduced into the tissue of interest in vivo or ex vivo by a variety of methods. In some embodiments of the invention, the nucleic acids of the invention are introduced to cells by such methods as microinjection, calcium phosphate precipitation, liposome fusion, or biolistics. In further embodiments, the nucleic acids are taken up directly by the tissue of interest.

In some embodiments of the invention, the nucleic acids of the invention are administered ex vivo to cells or tissues explanted from a patient, then returned to the patient. Examples of ex vivo administration of therapeutic gene constructs include Arteaga et al., Cancer Research 56(5):1098-1103 (1996); Nolta et al., Proc Natl. Acad. Sci. USA 93(6):2414-9 (1996); Koc et al., Seminars in Oncology 23 (1):46-65 (1996); Raper et al., Annals of Surgery 223(2):116-26 (1996); Dalesandro et al., J. Thorac. Cardi. Surg., 11(2):416-22 (1996); and Makarov et al., Proc. Natl. Acad. Sci. USA 93(1):402-6 (1996).

It is noted that many of the sequences described herein are publicly available in GenBank, which is the NIH genetic sequence database, an annotated collection of all publicly available DNA sequences (Nucleic Acids Research Jan. 1, 1998;26(1): 1-7).

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

Table 1 below indicates genes that demonstrate change in expression with aging. “CloneID” refers to the refers to the IMAGE Consortium library clone identification number. “DBID” is the Database ID, i.e., the database that lists the gene to which the clone has the highest homology: GB (Genbank), SP (Swissprot), or UG (Unigene). “GeneID” is the gene identification number in the database indicated in the “DBID” listing. “Species” refers to the species from which the GeneID was obtained. “5′ESTID” indicates the actual 5′ sequence ocrresponding to the clone ID. “3′ESTID” indicates the actual 3′ sequence corresponding to the clone ID. The “age-correlated change in expression” indicates whether gene expression is increased or decreased with aging, i.e., whether the expression level is increased or decreased in tissues that are obtained from old, i.e. aged, vs. young individuals.

TABLE 1

| | age correlated | | | | | | |
|---|---|---|---|---|---|---|---|
| GeneName | change in expression | CloneID | DBID | GeneID | Species | 5ESTID | 3 ESTID |
| HYPOTHETICAL 80.8 KD | downregulated with | 364444 | SP | P34588 | C. elegans | AA022764 | AA022664 |
| PROTEIN ZC21.4 IN | aging in muscle, | | | | | | |
| CHROMOSOME | liver, testis | | | | | | |
| EPIDERMAL 67-KDA TYPE II | upregulated with | 196022 | GB | M10938 | H. sapiens | R89443 | R89353 |
| KERATIN | aging in skin | | | | | | |
| DNA PRIMASE (SUBUNIT | downregulated with | 294461 | GB | X74330 | H. sapiens | W01522 | N70990 |
| P48) | aging in muscle, | | | | | | |
| | adrenal, testis | | | | | | |
| VRK2 PROTEIN KINASE | upregulated with | 197719 | GB | AB00450 | H. sapiens | R93692 | R94539 |
| | aging in skin | | | | | | |
| GTP-BINDING PROTEIN RAD | upregulated in liver | 238871 | GB | L24564 | H. sapiens | H64507 | H64508 |
| | and muscle with | | | | | | |
| | aging | | | | | | |
| XP-C REPAIR | upregulated with | 502671 | GB | D21090 | H. sapiens | AA125909 | AA127094 |
| COMPLEMENTING PROTEIN | aging in heart, | | | | | | |
| (P58/HHR23B) | skin, liver | | | | | | |
| ARGININOSUCCINATE | upregulated with | 626995 | GB | X01630 | H. sapiens | AA190498 | AA190840 |
| SYNTHETASE | aging in liver | | | | | | |
| CYTOCHROME C OXIDASE | upregulated with | 530049 | GB | X54691 | M. musculus | AA070078 | AA070648 |
| POLYPEPTIDE IV PRECURSOR | aging in liver, | | | | | | |
| | heart, skin | | | | | | |
| RAS-RELATED YPT1 PROTEIN | downregulated with | 363872 | GB | Y00094 | M. musculus | AA021068 | AA020983 |
| | aging in lung | | | | | | |
| RIBOSOMAL PROTEIN S15A | upregulated with | 624563 | GB | X62691 | H. sapiens | AA188457 | AA187332 |
| | aging in multiple | | | | | | |
| | tissues | | | | | | |
| PROTEIN KINASE C | upregulated with | 75415 | GB | U27143 | H. sapiens | T57609 | T57556 |
| INHIBITOR-I CDNA | aging in liver, | | | | | | |
| | lung, skin, heart | | | | | | |
| MYOSIN REGULATORY LIGHT | upregulated with | 124028 | GB | U26162 | H. sapiens | R02784 | R02785 |
| CHAIN | aging in heart, | | | | | | |
| | lung, muscle | | | | | | |
| KIDNEY-DERIVED ASPARTIC | upregulated with | 531607 | GB | D88899 | M. musculus | AA074707 | AA074174 |
| PROTEASE-LIKE PROTEIN | aging in skin, | | | | | | |
| | muscle, heart, liver | | | | | | |
| CYTOCHROME C OXIDASE | upregulated with | 123564 | GB | U90915 | H. sapiens | R01473 | R00817 |
| SUBUNIT IV | aging in liver, | | | | | | |
| | muscle | | | | | | |
| SPARC/OSTEONECTIN | upregulated with | 41677 | GB | J03040 | H. sapiens | R52908 | R67276 |
| | aging in fibroblasts | | | | | | |
| CAMP-DEPENDENT 3′,5′- | upregulated with | 46315 | GB | M97515 | H. sapiens | H09278 | H09279 |
| CYCLIC PHOSPHODIESTERASE | aging in colon, | | | | | | |
| 4B | muscle, heart, | | | | | | |
| | fibroblasts | | | | | | |
| INTERFERON-GAMMA INDUCED | upregulated with | 491243 | GB | X02530 | H. sapiens | AA150307 | AA152305 |
| PROTEIN PRECURSOR | aging in heart, | | | | | | |
| | muscle | | | | | | |
| PUTATIVE SURFACE | upregulated with | 505491 | GB | L48984 | H. sapiens | AA147452 | AA156461 |
| GLYCOPROTEIN PRECURSOR | aging in testis, | | | | | | |
| | colon, liver | | | | | | |
| CYCLIN G1 | upregulated with | 32322 | GB | X77794 | H. sapiens | R17447 | R42794 |
| | aging in testis, | | | | | | |
| | colon, liver | | | | | | |
| THYMOSIN BETA-4 | upregulated with | 594922 | GB | X02493 | H. sapiens | AA172262 | AA172027 |
| | aging in skin, lung, | | | | | | |
| | liver, fibroblasts | | | | | | |
| TRANSFORMING PROTEIN | upregulated with | 562742 | GB | X02751 | H. sapiens | AA111839 | AA086470 |
| P21/N-RAS | aging in liver | | | | | | |
| SERINE/THREONINE PROTEIN | upregulated with | 344639 | GB | Y10032 | H. sapiens | W73284 | W73229 |
| KINASE SGK | aging in heart, | | | | | | |
| | adrenal, | | | | | | |
| | fibroblasts, adrenal | | | | | | |
| 3-BETA HYDROXY-5-ENE | downregulated with | 428106 | GB | X55997 | H. sapiens | AA001626 | AA001627 |
| STEROID DEHYDROGENASE | aging in adrenal, | | | | | | |
| TYPE I | skin, liver, colon | | | | | | |
| MALATE DEHYDROGENASE, | upregulated with | 362135 | GB | D55654 | H. sapiens | AA001118 | AA000981 |
| CYTOPLASMIC | aging in colon, | | | | | | |
| | adrenal, heart, | | | | | | |
| | skin, liver | | | | | | |
| UNKNOWN | elevated (1.5) in | 213303 | | | | H71961 | |
| | multiple tissues | | | | | | |
| | with aging | | | | | | |
| UNKNOWN | elevated (1.5) in | 322437 | | | | | |
| | multiple tissues | | | | | | |
| | with aging | | | | | | |
| T-CELL RECEPTOR BETA 2 | elevated (1.5) in | 306841 | GB | X01411 | H. sapiens | | N91921 |
| CHAIN V-J-C (HPB-ALL) | multiple tissues | | | | | | |
| | with aging | | | | | | |
| LINE-1 REVERSE | elevated (1.5) in | 490962 | SP | P08547 | H. sapiens | AA120840 | AA120841 |
| TRANSCRIPTASE HOMOLOG | multiple tissues | | | | | | |
| | with aging | | | | | | |
| DNA PRIMASE (SUBUNIT | elevated (1.5) in | 294461 | GB | X74330 | H. sapiens | W01522 | N70990 |
| P48) | multiple tissues | | | | | | |
| | with aging | | | | | | |
| UNKNOWN | elevated (1.5) in | 205578 | | | | H58170 | H58171 |
| | multiple tissues | | | | | | |
| SNAP-23 | elevated (1.5) in | 248856 | GB | U55936 | H. sapiens | H82169 | H82068 |
| | multiple tissues | | | | | | |
| PRECURSOR OF P100 SERINE | elevated (1.5) in | 197757 | GB | D17525 | H. sapiens | R93502 | R93503 |
| PROTEASE OF RA-REACTIVE | multiple tissues | | | | | | |
| F | with aging | | | | | | |
| CARBONIC ANHYDRASE II | elevated (1.5) in | 120247 | GB | Y00339 | H. sapiens | T95737 | T95634 |
| | multiple tissues | | | | | | |
| | with aging | | | | | | |
| ATP-DEPENDENT RNA | | 428340 | GB | Y10658 | H. sapiens | AA005421 | AA005154 |
| HELICASE A | | | | | | | |
| ADP-RIBOSYLATION FACTOR | decreased (1.5) in | 119650 | GB | M84326 | H. sapiens | T96495 | T96412 |
| 1 | multiple tissues | | | | | | |
| | with aging | | | | | | |
| DIHYDROOROTASE AND | decreased (1.5) in | 569048 | GB | D78586 | H. sapiens | AA149106 | AA149107 |
| ASPARTATE | multiple tissues | | | | | | |
| TRANSCARBAMYLASE (CAD | with aging | | | | | | |
| BETA-2-MICROGLOBULIN | decreased (1.5) in | 300966 | GB | M17987 | H. sapiens | W07742 | N80682 |
| | multiple tissues | | | | | | |
| | with aging | | | | | | |
| CALMODULIN | decreased (1.5) in | 529486 | GB | M27319 | H. sapiens | AA070961 | AA070962 |
| | multiple tissues | | | | | | |
| | with aging | | | | | | |
| FUNGAL STEROL-C5- | decreased (1.5) in | 73104 | GB | D85181 | H. sapiens | T56570 | T56419 |
| DESATURASE HOMOLOG | multiple tissues | | | | | | |
| | with aging | | | | | | |
| RIBOSOMAL PROTEIN L3 | decreased (1.5) in | 62173 | GB | X73460 | H. sapiens | T40250 | T41111 |
| | multiple tissues | | | | | | |
| | with aging | | | | | | |
| PROTEIN KINASE C IOTA | decreased (1.5) in | 71622 | GB | L33881 | H. sapiens | T57957 | T57875 |
| ISOFORM | multiple tissues | | | | | | |
| | with aging | | | | | | |
| HYPOTHETICAL 13.5 KD | decreased (1.5) in | 531433 | SP | P28836 | Y. enterocolotica | | AA074064 |
| PROTEIN | multiple tissues | | | | | | |
| | with aging | | | | | | |
| TRANSLATIONALLY | decreased (1.5) in | 143025 | GB | X16064 | H. sapiens | R71275 | R71226 |
| CONTROLLED TUMOR PROTEIN | multiple tissues | | | | | | |
| | with aging | | | | | | |
| CHIMERA | decreased (1.5) in | 308340 | | | | W31301 | N93750 |
| | multiple tissues | | | | | | |
| | with aging | | | | | | |
| RIBOSOMAL PROTEIN L22 | decreased (1.5) in | 526399 | GB | D17653 | M. musculus | AA121102 | AA122354 |
| | multiple tissues | | | | | | |
| | with aging | | | | | | |
| EXTRACELLULAR PROTEIN | decreased (1.5) in | 69280 | GB | U03877 | H. sapiens | T58265 | T54343 |
| (S1-5) | multiple tissues | | | | | | |
| | with aging | | | | | | |
| HYPOTHETICAL 30.8 KD | decreased (1.5) in | 593061 | GB | U79274 | H. sapiens | AA158727 | AA158728 |
| PROTEIN | multiple tissues | | | | | | |
| | with aging | | | | | | |
| PHOSPHATIDYLETHANOLAMINE | decreased (1.5) in | 510327 | GB | X75252 | H. sapiens | AA053746 | AA053582 |
| BINDING PROTEIN | multiple tissues | | | | | | |
| | with aging | | | | | | |
| GLUCOSE TRANSPORTER | decreased (1.5) in | 116784 | GB | K03195 | H. sapiens | T89560 | T89470 |
| | multiple tissues | | | | | | |
| | with aging | | | | | | |
| UNKNOWN | increased in many | 273629 | | | | N46301 | N36987 |
| | tissues with aging | | | | | | |
| ARGINYL-TRNA SYNTHETASE, | Increased in many | 364902 | SP | P38714 | S. cerevisiae | | AA024496 |
| MITOCHONDRIAL PRECURSOR | tissues with aging | | | | | | |
| ICAM-3 | increased in many | 109950 | GB | X69819 | H. sapiens | T84198 | T88815 |
| | tissues with aging | | | | | | |
| DIPHOSPHOMEVALONATE | increased in many | 173661 | GB | U49260 | H. sapiens | H22519 | H22520 |
| DECARBOXYLASE | tissues with aging | | | | | | |
| DI-N-ACETYLCHITOBIASE | increased in many | 321723 | GB | M95767 | H. sapiens | W33050 | W35203 |
| | tissues with aging | | | | | | |
| LIGHT-MEDIATED | increased in many | 301875 | UG | 42140 | A. thaliana | W17171 | N92489 |
| DEVELOPMENT PROTEIN DET1 | tissues with aging | | | | | | |
| PERIPHERAL PLASMA | increased in many | 223193 | GB | AF035582 | H. sapiens | H85584 | H85585 |
| MEMBRANE PROTEIN CASK | tissues with aging | | | | | | |
| TISSUE FACTOR PRECURSOR | increased in many | 529043 | GB | M27436 | H. sapiens | AA064886 | AA064809 |
| | tissues with aging | | | | | | |
| REPETITIVE | increased in many | 195977 | | | H. sapiens | R91896 | R92731 |
| | tissues with aging | | | | | | |
| TRANSCRIPTION FACTOR SP2 | increased in many | 770397 | GB | D28588 | H. sapiens | AA42749B | AA430659 |
| | tissues with aging | | | | | | |
| POLY(A) POLYMERASE | increased in many | 511066 | GB | X76770 | H. sapiens | AA099804 | AA100296 |
| | tissues with aging | | | | | | |
| U2 SNRNP AUXILIARY | increased in many | 325627 | GB | M96982 | H. sapiens | W51842 | W51814 |
| FACTOR SMALL SUBUNIT | tissues with aging | | | | | | |
| KIAA0397 | increased in many | 362603 | GB | AB007857 | H. sapiens | AA017335 | AA017061 |
| | tissues with aging | | | | | | |
| VASOPRESSIN V1A RECEPTOR | decreased in many | 155723 | GB | S73899 | H. sapiens | R72131 | R72081 |
| | tissues with aging | | | | | | |
| PROSTAGLANDIN E2 | decreased in many | 266525 | GB | L28175 | H. sapiens | N31182 | N22708 |
| RECEPTOR EP2 SUBTYPE | tissues with aging | | | | | | |
| RETINOIC ACID RECEPTOR | decreased in many | 358433 | GB | U38480 | H. sapiens | N96098 | W96099 |
| RXR-GAMMA | tissues with aging | | | | | | |
| ALPHA1-FETOPROTEIN | decreased in many | 246872 | GB | U93558 | H. sapiens | N59515 | N59115 |
| TRANSCRIPTION FACTOR | tissues with aging | | | | | | |
| SHORT VARI | | | | | | | |
| ISLET AMYLOID | decreased in many | 328284 | GB | J04422 | H. sapiens | W39293 | W31865 |
| POLYPEPTIDE (HIAPP) | tissues with aging | | | | | | |
| MHC CLASS I PROMOTER | decreased in many | 66621 | GB | X65463 | H. sapiens | T67174 | T67173 |
| BINDING PROTEIN | tissues with aging | | | | | | |
| PROTEIN TYROSINE KINASE | interesting genes | 41097 | GB | X75208 | H. sapiens | R56713 | R56867 |
| RECEPTOR HEK2 | broadly down during | | | | | | |
| | aging | | | | | | |
| SERINE KINASE | decreased in many | 246240 | GB | U09564 | H. sapiens | N77083 | N59394 |
| | tissues with aging | | | | | | |
| 5-HYDROXYTRYPTAMINE 3 | decreased in many | 127204 | GB | S82612 | H. sapiens | R08225 | R08170 |
| RECEPTOR PRECURSOR | tissues with aging | | | | | | |
| TGF-BETAIIR ALPHA | decreased in many | 240950 | GB | D50683 | H. sapiens | H90996 | H90886 |
| | tissues with aging | | | | | | |
| NUCLEAR RECEPTOR ROR- | decreased in many | 382922 | GB | Y08639 | H. sapiens | AA084457 | |
| BETA | tissues with aging | | | | | | |
| ENDOTHELIN-1 RECEPTOR. | decreased in many | 504085 | GB | X61950 | H. sapiens | AA131759 | AA132863 |
| | tissues with aging | | | | | | |
| K+ CHANNEL BETA SUBUNIT | decreased in many | 360213 | GB | L39833 | H. sapiens | AA013094 | AA013095 |
| | tissues with aging | | | | | | |
| C-FMS PROTO-ONCOGENE | decreased in many | 123502 | GB | X03663 | H. sapiens | R00726 | R00727 |
| | tissues with aging | | | | | | |
| PROSTACYCLIN RECEPTOR | decreased in many | 774146 | GB | D29634 | H. sapiens | AA429812 | AA428147 |
| | tissues with aging | | | | | | |
| EAR-1R | decreased in many | 259299 | GB | D16815 | H. sapiens | N41813 | N32859 |
| | tissues with aging | | | | | | |
| AMP-ACTIVATED PROTEIN | decreased in many | 300137 | GB | AJ224538 | H. sapiens | W07176 | N78582 |
| KINASE BETA 2 SUBUNIT. | tissues with aging | | | | | | |
| ATRIAL NATRIURETIC | decreased in many | 152523 | GB | X15357 | H. sapiens | R46850 | R46756 |
| PEPTIDE RECEPTOR A | tissues with aging | | | | | | |
| PRECURSOR | | | | | | | |
| PROSTANOID FP RECEPTOR | interesting genes | 151011 | GB | L24470 | H. sapiens | H02113 | H02015 |
| | broadly down during | | | | | | |
| | aging | | | | | | |
| CHLORIDE CHANNEL PROTEIN | decreased in many | 120287 | GB | X83378 | H. sapiens | T97200 | T97201 |
| 6 | tissues with aging | | | | | | |
| INTERFERON-ALPHA- | decreased (1.5) in | 587600 | GB | X67325 | H. sapiens | AA132995 | AA132959 |
| INDUCIBILE P27 | multiple tissues | | | | | | |
| | with aging | | | | | | |
| GAMMA-AMINOBUTYRIC-ACID | decreased (1.5) in | 28218 | GB | X15376 | H. sapiens | R13309 | R40790 |
| RECEPTOR GAMMA-2 SUBUNIT | multiple tissues | | | | | | |
| P | with aging | | | | | | |
| MITOTIC CONTROL PROTEIN | decreased (1.5) in | 546474 | GB | M74094 | S. pombe | AA080953 | AA081429 |
| (DIS3+) | multiple tissues | | | | | | |
| | with aging | | | | | | |
| RIBOSOMAL PROTEIN S4 × | decreased (1.5) in | 530081 | GB | M58458 | H. sapiens | AA070713 | AA070510 |
| ISOFORM | multiple tissues | | | | | | |
| | with aging | | | | | | |
| HLA CLASS II | decreased (1.5) in | 79970 | GB | X03067 | H. sapiens | T63385 | T63529 |
| HISTOCOMPATIBILITY | multiple tissues | | | | | | |
| ANTIGEN, DP (W2) BE | with aging | | | | | | |
| RIBOSOMAL PROTEIN S6 | decreased (1.5) in | 587780 | SP | P51812 | H. sapiens | AA134358 | AA134359 |
| KINASE II ALPHA 2 | multiple tissues | | | | | | |
| | with aging | | | | | | |
| LAMININ RECEPTOR | decreased (1.5) in | 41669 | GB | X15005 | H. sapiens | R52870 | R66451 |
| | multiple tissues | | | | | | |
| | with aging | | | | | | |
| NEUROENDOCRINE/BETA- | decreased (1.5) in | 36581 | GB | M83566 | H. sapiens | R25307 | R46658 |
| CELL-TYPE CALCIUM | multiple tissues | | | | | | |
| CHANNEL ALPH | with aging | | | | | | |
| RIBOSOMAL PROTEIN S6 | decreased (1.5) in | 269279 | GB | M20020 | H. sapiens | N35861 | N24040 |
| | multiple tissues | | | | | | |
| | with aging | | | | | | |
| APRIL PROTEIN | decreased (1.5) in | 509572 | GB | Y07969 | H. sapiens | AA056627 | AA056602 |
| | multiple tissues | | | | | | |
| | with aging | | | | | | |
| SKELETAL MUSCLE 165KD | decreased (1.5) in | 300219 | GB | X69089 | H. sapiens | W07234 | N78805 |
| PROTEIN | multiple tissues | | | | | | |
| | with aging | | | | | | |
| IMITOCHONDRIAL DNA | decreased (1.5) in | 755597 | GB | X93334 | H. sapiens | AA419285 | AA419250 |
| | multiple tissues | | | | | | |
| | with aging | | | | | | |
| KIAA0027 | decreased (1.5) in | 730828 | GB | D25217 | H. sapiens | AA416907 | AA417008 |
| | multiple tissues | | | | | | |
| | with aging | | | | | | |
| RPL13-2 PSEUDOGENE | decreased (1.5) in | 587452 | GB | U72513 | H. sapiens | AA132643 | AA132537 |
| | multiple tissues | | | | | | |
| | with aging | | | | | | |
| RIBOSOMAL PROTEIN P0, | decreased (1.5) in | 362986 | GB | M17885 | H. sapiens | AA019139 | AA019059 |
| ACIDIC | multiple tissues | | | | | | |
| | with aging | | | | | | |
| RIBOSOMAL PROTEIN S16 | decreased (1.5) in | 126697 | GB | M60854 | H. sapiens | R07057 | R07019 |
| | multiple tissues | | | | | | |
| | with aging | | | | | | |
| RIBOSOMAL PROTEIN L19 | decreased (1.5) in | 529388 | GB | X63527 | H. sapiens | AA070761 | AA070762 |
| | multiple tissues | | | | | | |
| | with aging | | | | | | |
| PUT. RING PROTEIN | decreased (1.5) in | 509760 | GB | Y07828 | H. sapiens | AA054421 | AA054321 |
| | multiple tissues | | | | | | |
| | with aging | | | | | | |
| GLYCOGEN PHOSPHORYLASE, | decreased (1.5) in | 561726 | GB | X03031 | H. sapiens | AA100564 | AA086351 |
| MUSCLE FORM | multiple tissues | | | | | | |
| | with aging | | | | | | |
| RIBOSOMAL PROTEIN L7A | decreased (1.5) in | 300042 | GB | M36072 | H. sapiens | W07149 | N91538 |
| | multiple tissues | | | | | | |
| | with aging | | | | | | |
| COFILIN, NON-MUSCLE | decreased (1.5) in | 484557 | GB | X95404 | H. sapiens | AA036983 | AA036984 |
| ISOFORM | multiple tissues | | | | | | |
| | with aging | | | | | | |
| STRESS RESPONSIVE | decreased (1.5) in | 120015 | GB | U60207 | H. sapiens | T94961 | T95014 |
| SERINE/THREONINE PROTEIN | multiple tissues | | | | | | |
| KINASE | with aging | | | | | | |
| HEAT SHOCK PROTEIN | decreased (1.5) in | 33800 | GB | X51758 | H. sapiens | R24850 | R44553 |
| HSP70B′ | multiple tissues | | | | | | |
| | with aging | | | | | | |
| LEUKOCYTE ELASTASE | decreased (1.5) in | 205836 | GB | M34379 | H. sapiens | H58275 | H58668 |
| PRECURSOR | multiple tissues | | | | | | |
| | with aging | | | | | | |
| CD63 ANTIGEN | decreased (1.5) in | 471872 | GB | M58485 | H. sapiens | AA035756 | AA035150 |
| | multiple tissues | | | | | | |
| | with aging | | | | | | |
| CHIMERA | decreased (1.5) in | 155283 | | | | R70457 | R70401 |
| | multiple tissues | | | | | | |
| | with aging | | | | | | |
| RIBOSOMAL PROTEIN L12 | decreased (1.5) in | 175532 | GB | L06505 | H. sapiens | H41199 | H41200 |
| | multiple tissues | | | | | | |
| | with aging | | | | | | |
| LYSOSOME-ASSOCIATED | decreased (1.5) in | 529056 | GB | J04182 | H. sapiens | AA064889 | AA064821 |
| MEMBRANE GLYCOPROTEIN 1 | multiple tissues | | | | | | |
| PRECUR | with aging | | | | | | |
| RIBOSOMAL PROTEIN 526 | decreased (1.5) in | 427950 | GB | X69654 | H. sapiens | | AA001821 |
| | multiple tissues | | | | | | |
| | with aging | | | | | | |
| RIBOSOMAL PROTEIN L41 | decreased (1.5) in | 300040 | GB | Z12962 | H. sapiens | W07148 | N91537 |
| | multiple tissues | | | | | | |
| | with aging | | | | | | |
| BETA CRYSTALLIN A3 | decreased (1.5) in | 609505 | GB | U59058 | H. sapiens | AA180075 | AA180156 |
| | multiple tissues | | | | | | |
| | with aging | | | | | | |
| FLAVOPROTEIN SUBUNIT OF | decreased (1.5) in | 544767 | GB | D30648 | H. sapiens | AA074982 | AA074907 |
| COMPLEX II | multiple tissues | | | | | | |
| | with aging | | | | | | |
| CX3C CHEMOKINE PRECURSOR | decreased (1.5) in | 29324 | GB | U84487 | H. sapiens | R14548 | R41210 |
| | multiple tissues | | | | | | |
| | with aging | | | | | | |
| Emerin (EDMD) | decreased (1.5) in | 321250 | GB | X82434 | H. sapiens | W52798 | AA037393 |
| | multiple tissues | | | | | | |
| | with aging | | | | | | |
| PROTEASOME SUBUNIT X | decreased (1.5) in | 486166 | GB | D29011 | H. sapiens | AA040693 | AA040694 |
| | multiple tissues | | | | | | |
| | with aging | | | | | | |
| GUANINE NUCLEOTIDE- | decreased (1.5) in | 23019 | GB | X07036 | H. sapiens | T74985 | R43581 |
| BINDING PROTEIN G(S), | multiple tissues | | | | | | |
| ALPHA SUB | with aging | | | | | | |
| UNKNOWN | decreased (1.5) in | 25296 | | | | R11712 | R17687 |
| | multiple tissues | | | | | | |
| | with aging | | | | | | |
| AQUAPORIN-CHIP | decreased (1.5) in | 628768 | GB | U41518 | H. sapiens | AA194331 | AA194307 |
| | multiple tissues | | | | | | |
| | with aging | | | | | | |
| ANTI-ONCOGENE | decreased (1.5) in | 159537 | GB | M98056 | H. sapiens | H16114 | H15813 |
| | multiple tissues | | | | | | |
| | with aging | | | | | | |
| XE169 | decreased (1.5) in | 322859 | GB | L25270 | H. sapiens | W39589 | W44939 |
| | multiple tissues | | | | | | |
| | with aging | | | | | | |
| DNA-BINDING PROTEIN | decreased (1.5) in | 62339 | GB | D13891 | H. sapiens | T40351 | T41210 |
| INHIBITOR ID-2 | multiple tissues | | | | | | |
| | with aging | | | | | | |
| HYDROXYSTEROID | decreased (1.5) in | 591387 | GB | U92315 | H. sapiens | AA159425 | AA159317 |
| SULFOTRANSFERASE HSST2B | multiple tissues | | | | | | |
| | with aging | | | | | | |
| LEUKOCYTE IGG RECEPTOR | decreased (1.5) in | 545469 | GB | J04162 | H. sapiens | AA079871 | AA079872 |
| (FC-GAMNA-R) | multiple tissues | | | | | | |
| | with aging | | | | | | |
| ZINC FINGER PROTEIN 91 | decreased (1.5) in | 31631 | SP | Q05481 | H. sapiens | R22697 | R43403 |
| | multiple tissues | | | | | | |
| | with aging | | | | | | |
| KU AUTOANTIGEN P70 | decreased (1.5) in | 525718 | GB | S38729 | H. sapiens | AA069845 | AA069798 |
| SUBUNIT | multiple tissues | | | | | | |
| | with aging | | | | | | |
| S19 RIBOSOMAL PROTEIN | decreased (1.5) in | 230363 | GB | M81757 | H. sapiens | H80971 | H80870 |
| | multiple tissues | | | | | | |
| | with aging | | | | | | |
| ADVILLIN | decreased (1.5) in | 281837 | GB | AF041449 | H. sapiens | N54096 | N51826 |
| | multiple tissues | | | | | | |
| | with aging | | | | | | |
| HYPOTHETICAL 48.5 KD | decreased (1.5) in | 531710 | GB | U79241 | H. sapiens | AA074544 | |
| PROTEIN | multiple tissues | | | | | | |
| | with aging | | | | | | |
| GLUTAMINYL-TRNA | decreased (1.5) in | 544986 | GB | X54326 | H. Sapiens | AA075639 | AA075640 |
| SYNTHETASE | multiple tissues | | | | | | |
| | with aging | | | | | | |
| SPOP | decreased (1.5) in | 75469 | GB | AJ000644 | H. sapiens | T58988 | T57634 |
| | multiple tissues | | | | | | |
| | with aging | | | | | | |
| SPHINGOLIPID ACTIVATOR | decreased (1.5) in | 360383 | GB | D00422 | H. sapiens | AA015630 | AA015631 |
| PROTEINS | multiple tissues | | | | | | |
| | with aging | | | | | | |
| INTERFERON-ALPHA- | decreased (1.5) in | 238520 | GB | X67325 | H. sapiens | H64676 | H64574 |
| INDUCIBILE P27 | multiple tissues | | | | | | |
| | with aging | | | | | | |
| IROQUOIS-CLASS | elevated (5×) in | 152453 | GB | U90304 | H. sapiens | R46296 | R46202 |
| HOMEODOMAIN PROTEIN IRX- | multiple tissues | | | | | | |
| 2A | with aging | | | | | | |
| HIGH MOBILITY GROUP | elevated (5×) in | 190646 | GB | L17131 | H. sapiens | H38585 | H38829 |
| PROTEIN HMG-I | multiple tissues | | | | | | |
| | with aging | | | | | | |
| TESTIN 2 | elevated (5×) in | 233870 | SP | P47226 | M. musculus | H67804 | H68077 |
| | multiple tissues | | | | | | |
| | with aging | | | | | | |
| EARLY GROWTH RESPONSE | elevated (5×) in | 362693 | GB | J04076 | H. sapiens | AA018140 | AA018188 |
| PROTEIN 2 (EGR2) | multiple tissues | | | | | | |
| | with aging | | | | | | |
| HYPOTHETICAL 25.7 KD | elevated (5×) in | 258613 | SP | P38829 | S. cerevisiae | N57306 | N32208 |
| PROTEIN IN MSH1-EPT1 | multiple tissues | | | | | | |
| INTERGEN | with aging | | | | | | |
| TYROSINE KINASE | elevated (5×) in | 526536 | GB | Z50150 | H. sapiens | AA128438 | |
| ACTIVATOR PROTEIN 1 | multiple tissues | | | | | | |
| (TKA-1) | with aging | | | | | | |
| NEURON-SPECIFIC RNA | elevated (5×) in | 42615 | GB | S69265 | H. sapiens | R61633 | R60966 |
| RECOGNITION MOTIFS | multiple tissues | | | | | | |
| (RRMS)-CONT | with aging | | | | | | |
| CD45-BINDING PROTEIN | elevated (5×) in | 324712 | GB | X97267 | H. sapiens | W47352 | W47353 |
| | multiple tissues | | | | | | |
| | with aging | | | | | | |
| UNKNOWN | elevated (5×) in | 322409 | | | | | |
| | multiple tissues | | | | | | |
| | with aging | | | | | | |
| MELANIN-CONCENTRATING | elevited (5×) in | 773341 | GB | M57703 | H. sapiens | AA425639 | AA425440 |
| HORMONE PRECURSOR | multiple tissues | | | | | | |
| SERINE-THREONINE KINASE | elevated (5×) in | 269812 | GB | U02890 | R. novegicus | N40091 | N27153 |
| | multiple tissues | | | | | | |
| | with aging | | | | | | |
| MULTIPLE EXOSTOSIS-LIKE | elevated (5×) in | 26442 | GB | U67191 | H. sapiens | R12464 | R37350 |
| PROTEIN (EXTL) | multiple tissues | | | | | | |
| | with aging | | | | | | |
| PROTEIN TRANSLATION | elevated (5×) in | 362880 | GB | L26247 | H. sapiens | AA019470 | AA019535 |
| FACTOR SUI1 HOMOLOG | multiple tissues | | | | | | |
| | with aging | | | | | | |
| UNC-50 RELATED PROTEIN | elevated (5×) in | 489814 | GB | U96638 | R. novegicus | AA099343 | AA102090 |
| | multiple tissues | | | | | | |
| | with aging | | | | | | |
| VASCULAR ENDOTHELIAL | elevated (5×) in | 116501 | GB | X61656 | H. sapiens | T91460 | T91369 |
| GROWTH FACTOR RECEPTOR 2 | multiple tissues | | | | | | |
| PREC | with aging | | | | | | |
| MICROTUBULE-ASSOCIATED | elevated (5×) in | 21951 | SP | P46821 | H. sapiens | T66206 | T66142 |
| PROTEIN 1B | multiple tissues | | | | | | |
| | with aging | | | | | | |
| F-ACTIN CAPPING PROTEIN | elevated (5×) in | 563878 | GB | U03271 | H. sapiens | AA101401 | AA101402 |
| BETA SUBUNIT | multiple tissues | | | | | | |
| | with aging | | | | | | |
| RIBOSOMAL PROTEIN S18 | decreased (5×) in | 590351 | GB | X69150 | H. sapiens | AA147912 | AA147855 |
| | multiple tissues | | | | | | |
| | with aging | | | | | | |
| RIBOSOMAL PROTEIN S15A | decreased (5×) in | 340492 | GB | X62691 | H. sapiens | W52758 | W52024 |
| | multiple tissues | | | | | | |
| | with aging | | | | | | |
| NADH-UBIQUINONE | decreased (5×) in | 38400 | UG | 12283 | B. taurus | R35603 | |
| OXIDOREDUCTASE B15 | multiple tissues | | | | | | |
| SUBUNIT | with aging | | | | | | |
| TYROSINE KINASE RET | decreased (5×) in | 53190 | GB | M16029 | H. sapiens | T66742 | T66741 |
| | multiple tissues | | | | | | |
| | with aging | | | | | | |
| KIAA0040 | decreased (5×) in | 488634 | GB | D25539 | H. sapiens | AA044572 | AA044894 |
| | multiple tissues | | | | | | |
| | with aging | | | | | | |
| METABOTROPIC GLUTAMATE | decreased (5×) in | 32991 | GB | L35318 | H. sapiens | R19103 | R44770 |
| RECEPTOR TYPE II (GLUR2) | multiple tissues | | | | | | |
| | with aging | | | | | | |
| GLIA MATURATION FACTOR | decreased (5×) in | 505492 | GB | AB001993 | H. sapiens | AA147584 | AA156455 |
| HOMOLOGOUS PROTEIN | multiple tissues | | | | | | |
| | with aging | | | | | | |
| TRANSCRIPTION FACTOR AP- | decreased (5×) in | 363520 | GB | X95694 | H. sapiens | AA019703 | AA019704 |
| 2 BETA | multiple tissues | | | | | | |
| | with aging | | | | | | |
| MICROTUBULE-ASSOCIATED | decreased (5×) in | 363776 | GB | U01828 | H. sapiens | AA020894 | AA020782 |
| PROTEIN 2 (MAP2) | multiple tissues | | | | | | |
| | with aging | | | | | | |
| G1/S-SPECIFIC CYCLIN D2 | decreased (5×) in | 48206 | GB | D13639 | H. sapiens | H11231 | H11125 |
| | multiple tissues | | | | | | |
| | with aging | | | | | | |
| KIAA0054 | decreased (5×) in | 197358 | GB | D29677 | H. sapiens | R86774 | R86753 |
| | multiple tissues | | | | | | |
| | with aging | | | | | | |
| VACUOLAR ATP SYNTHASE | decreased (5×) in | 156211 | GB | M25809 | H. sapiens | R73401 | R73402 |
| SUBUNIT B, KIDNEY | multiple tissues | | | | | | |
| ISOFORM | with aging | | | | | | |
| PAPS SYNTHETASE | decreased (5×) in | 276543 | GB | Y10387 | H. sapiens | N48472 | N39112 |
| | multiple tissues | | | | | | |
| | with aging | | | | | | |
| 44.9 KDA PROTEIN C18B11 | decreased (5×) in | 664425 | GB | U67934 | H. sapiens | AA243115 | AA232194 |
| HOMOLOG | multiple tissues | | | | | | |
| | with aging | | | | | | |
| BREAST EPITHELIAL | decreased (5×) in | 156295 | GB | U58516 | H. sapiens | R72681 | R72612 |
| ANTIGEN BA46 | multiple tissues | | | | | | |
| | with aging | | | | | | |
| UNKNOWN | decreased (5×) in | 23141 | | | | T75364 | R39195 |
| | multiple tissues | | | | | | |
| | with aging | | | | | | |
| TRANSALDOLASE A | decreased (5×) in | 75898 | SP | P78258 | E. coli | | T59433 |
| | multiple tissues | | | | | | |
| | with aging | | | | | | |
| 5-HYDROXYTRYPTAMINE 2C | decreased (5×) in | 280371 | GB | U49516 | H. sapiens | N50321 | N47111 |
| RECEPTOR | multiple tissues | | | | | | |
| | with aging | | | | | | |
| C-ETS-2 PROTEIN | decreased (5×) in | 52650 | GB | J04102 | H. sapiens | H29776 | H29777 |
| | multiple tissues | | | | | | |
| | with aging | | | | | | |
| CORTICOSTEROID 11-BETA- | decreased (5×) in | 360247 | SP | P50172 | M. musculus | AA012839 | AA012823 |
| DEHYDROGENASE, ISOZYME 1 | multiple tissues | | | | | | |
| | with aging | | | | | | |
| RIBOSOMAL PROTEIN L36 | decreased (5×) in | 625243 | GB | X75895 | M. musculus | AA182966 | AA181098 |
| | multiple tissues | | | | | | |
| | with aging | | | | | | |
| TUMOR NECROSIS FACTOR | decreased (5×) in | 79742 | GB | L04270 | H. sapiens | T63190 | T62568 |
| RECEPTOR 2 RELATED | multiple tissues | | | | | | |
| PROTEIN | with aging | | | | | | |
| CHROMOSOME 15 MAD | decreased (5×) in | 741694 | GB | U59914 | H. sapiens | AA402939 | AA402014 |
| HOMOLOG SMAD6 | multiple tissues | | | | | | |
| | with aging | | | | | | |
| MYOSIN LIGHT CHAIN 2 | decreased (5×) in | 298706 | GB | M21812 | H. sapiens | W05048 | |
| | multiple tissues | | | | | | |
| | with aging | | | | | | |
| TAR RNA BINDTNG PROTEIN | decreased (5×) in | 156223 | GB | U08998 | H. sapiens | R73301 | R72841 |
| 2 (TRBP2) | multiple tissues | | | | | | |
| | with aging | | | | | | |
| METALLOTHIONEIN 1L (MT- | decreased (5×) in | 240883 | GB | X76717 | H. sapiens | H80890 | H80891 |
| 1L) | multiple tissues | | | | | | |
| | with aging | | | | | | |
| KIAA0210 | decreased (5×) in | 28572 | GB | D86965 | H. sapiens | R13386 | R40902 |
| | multiple tissues | | | | | | |
| | with aging | | | | | | |
| RIBOSOMAL PROTEIN S7 | decreased (5×) in | 73590 | GB | M77233 | H. sapiens | T55686 | T55604 |
| | multiple tissues | | | | | | |
| | with aging | | | | | | |
| MITOCHONDRIAL DNA | decreased (5×) in | 382815 | GB | X93334 | H. sapiens | AA069655 | AA069464 |
| | multiple tissues | | | | | | |
| | with aging | | | | | | |
| RIBOSOMAL PROTEIN P1, | decreased (5×) in | 530260 | GB | U29402 | M. musculus | AA083722 | AA111987 |
| ACIDIC | multiple tissues | | | | | | |
| | with aging | | | | | | |
| HETEROGENEOUS NUCLEAR | decreased (5×) in | 301063 | GB | L28010 | H. sapiens | W07799 | N81030 |
| RIBONUCLEOPROTEIN F | multiple tissues | | | | | | |
| | with aging | | | | | | |
| METALLOTHIONEIN ISOFORM | decreased (5×) in | 82154 | GB | V00594 | H. sapiens | T68901 | T68830 |
| 2 | multiple tissues | | | | | | |
| | with aging | | | | | | |
| FIBROPELLIN C PRECURSOR | decreased (5×) in | 25810 | SP | P49013 | S. purpuratus | R12231 | R39949 |
| | multiple tissues | | | | | | |
| | with aging | | | | | | |
| THYROID HORMONE- | decreased (5×) in | 82067 | GB | Y08409 | H. sapiens | T68776 | T68711 |
| INDUCIBLE HEPATIC | multiple tissues | | | | | | |
| PROTEIN | with aging | | | | | | |
| UNKNOWN | decreased (5×) in | 322339 | | | | | |
| | multiple tissues | | | | | | |
| | with aging | | | | | | |
| UNKNOWN | decreased (5×) in | 511952 | | | | | AA100674 |
| | multiple tissues | | | | | | |
| | with aging | | | | | | |
| INTERFERON ALPHA INDUCED | decreased in many | 682770 | GB | M97934 | H. sapiens | AA210865 | AA210708 |
| TRANSCRIPTIONAL | tissues with aging | | | | | | |
| ACTIVATOR | | | | | | | |
| MYOSIN LIGHT CHAIN 1, | decreased in many | 562485 | SP | P05976 | H. sapiens | AA112996 | AA086301 |
| SKELETAL MUSCLE ISOFORM | tissues with aging | | | | | | |
| PARATHYMOSIN | decreased in many | 82843 | GB | M24398 | H. sapiens | T69323 | T69249 |
| | tissues with aging | | | | | | |
| 26S PROTEASE REGULATORY | decreased in many | 624891 | GB | L02426 | H. sapiens | AA186816 | AA181913 |
| SUBUNIT 4 | tissues with aging | | | | | | |
| KIAA0027 | decreased in many | 730828 | GB | D25217 | H. sapiens | AA416907 | AA417008 |
| | tissues with aging | | | | | | |
| RIBOSOMAL PROTEIN L37A | decreased in many | 427959 | GB | L22154 | H. sapiens | AA002035 | AA001831 |
| | tissues with aging | | | | | | |
| PUTATIVE SURFACE | decreased in many | 69684 | GB | Z50022 | H. sapiens | T53634 | T53635 |
| GLYCOPROTEIN PRECURSOR | tissues with aging | | | | | | |
| UNKNOWN | decreased in many | 325012 | | | | W48628 | W48725 |
| | tissues with aging | | | | | | |
| RIBOSOMAL PROTEIN S3A | decreased in many | 510289 | GB | M84711 | H. sapiens | AA053622 | AA053175 |
| | tissues with aging | | | | | | |
| RNA-BINDING PROTEIN | decreased in many | 79035 | GB | AF021819 | H. sapiens | T61971 | T61908 |
| REGULATORY SUBUNIT | tissues with aging | | | | | | |
| C-1-TETRAHYDROFOLATE | decreased in many | 31861 | SP | P11586 | H. sapiens | R17310 | R41989 |
| SYNTHASE, CYTOPLASMIC | tissues with aging | | | | | | |
| POTASSIUM CHANNEL KV2.1 | decreased in many | 381974 | GB | L02840 | H. sapiens | AA063012 | AA063031 |
| | tissues with aging | | | | | | |
| RIBOSOMAL PROTEIN S25 | decreased in many | 470130 | GB | M64716 | H. sapiens | AA029957 | AA029958 |
| | tissues with aging | | | | | | |
| RIBOSOMAL PROTEIN S20 | decreased in many | 117268 | GB | L06498 | H. sapiens | T93722 | T96186 |
| (RPS20) | tissues with aging | | | | | | |
| MYOSIN HEAVY CHAIN, | decreased in many | 22140 | GB | M69181 | H. sapiens | T64807 | T72559 |
| NONMUSCLE TYPE A | tissues with aging | | | | | | |
| UNKNOWN | increased in many | 75494 | | | | T59305 | T57642 |
| | tissues with aging | | | | | | |
| RED CELL ANION EXCHANGER | increased in many | 195416 | GB | X77738 | H. sapiens | | R89603 |
| (EPB3, AE1, BAND 3) | tissues with aging | | | | | | |
| UEV1BS (UBE2V) | increased in many | 47435 | GB | U97280 | H. sapiens | H11281 | H11282 |
| | tissues with aging | | | | | | |
| UNKNOWN | increased in many | 281041 | | | | | N50902 |
| | tissues with aging | | | | | | |
| UNKNOWN | increased in many | 127049 | | | | | R07988 |
| | tissues with aging | | | | | | |
| U2 SNRNP AUXILIARY | increased in many | 509691 | GB | M96982 | H. sapiens | AA058483 | AA058364 |
| FACTOR SMALL SUBUNIT | tissues with aging | | | | | | |
| REPETITIVE | increased in many | 109841 | | | H. sapiens | T85153 | T88881 |
| | tissues with aging | | | | | | |
| PET112 PROTEIN PRECURSOR | increased in many | 80716 | UG | 11127 | S. cerevisiae | T63207 | T62957 |
| | tissues with aging | | | | | | |
| REPETITIVE | increased in many | 118754 | | | H. sapiens | T91659 | T93260 |
| | tissues with aging | | | | | | |
| EUKARYOTIC TRANSLATION | increased in many | 113597 | GB | U49436 | H. sapiens | T79280 | T79193 |
| INITIATION FACTOR 5 | tissues with aging | | | | | | |
| PLATELET ACTIVATING | decreased in many | 60403 | GB | M80436 | H. sapiens | T39278 | |
| FACTOR RECEPTOR | tissues with aging | | | | | | |
| HOMOSAPIENS ERK | decreased in many | 115767 | GB | L11284 | H. sapiens | T87961 | T87872 |
| ACTIVATOR KINASE (MEK1) | tissues with aging | | | | | | |
| ACTIVATED P21CDC42HS | decreased in many | 33211 | GB | L13738 | H. sapiens | R19138 | R44803 |
| KINASE (ACK) | tissues with aging | | | | | | |
| CASEIN KINASE I, GAMMA 2 | decreased in many | 346031 | GB | U89896 | H. sapiens | W77970 | W72092 |
| ISOFORM | tissues with aging | | | | | | |
| PROBABLE GLUTATHIONE | decreased in many | 66378 | UG | 12971 | C. elegans | T66886 | T66885 |
| REDUCTASE | tissues with aging | | | | | | |
| CYTOCHROME P450 | decreased in many | 114735 | GB | D12621 | H. sapiens | T85456 | T85359 |
| | tissues with aging | | | | | | |
| 2-OXOGLUTARATE | decreased in many | 108781 | SP | P07015 | E. coli | T77704 | T77882 |
| DEHYDROGENASE E1 | tissues with aging | | | | | | |
| COMPONENT | | | | | | | |
| SERINE/THREONINE- | increased in many | 43033 | GB | D86550 | H. sapiens | R60178 | R60179 |
| SPECIFIC PROTEIN KINASE | tissues with aging | | | | | | |
| MINIBRAIN | | | | | | | |
| LIPID-ACTIVATED, PROTEIN | increased in many | 550355 | GB | U33052 | H. sapiens | AA101793 | AA098980 |
| KINASE PRK2 | tissues with aging | | | | | | |
| PUTATIVE G PROTEIN- | increased in many | 42685 | GB | U66581 | H. sapiens | R59799 | R61341 |
| COUPLED RECEPTOR (GPR22) | tissues with aging | | | | | | |
| MYOTONIN-PROTEIN KINASE | increased in many | 345643 | GB | L19267 | H. sapiens | W76568 | W71999 |
| | tissues with aging | | | | | | |
| C PROTEIN-COUPLED | increased in many | 255333 | GB | X97879 | H. sapiens | | N23898 |
| RECEPTOR KINASE GRK4 | tissues with aging | | | | | | |
| N-METHYL-D-ASPARTATE | increased in many | 163879 | GB | U08106 | H. sapiens | | H39722 |
| RECEPTOR SUBUNIT (GRIN1) | tissues with aging | | | | | | |
| AMILORIDE-SENSITIVE | increased in many | 163045 | GB | X87159 | H. sapiens | H26938 | |
| SODIUM CHANNEL BETA- | tissues with aging | | | | | | |
| SUBUNIT | | | | | | | |
| N-FORMYLPEPTIDE RECEPTOR | increased in many | 117822 | GB | M60627 | H. sapiens | T90598 | T90501 |
| (FMLP-R26) | tissues with aging | | | | | | |
| CAMP-DEPENDENT PROTEIN | increased in many | 429142 | GB | X07767 | H. sapiens | AA005272 | AA005273 |
| KINASE, ALPHA-CATALYTIC | tissues with aging | | | | | | |
| SUB | | | | | | | |
| P64 BOVINE CHLORIDE | increased in many | 71956 | GB | Y12696 | H. sapiens | T52280 | T52201 |
| CHANNEL PEPTIDE HOMOLOG | tissues with aging | | | | | | |
| SODIUM CHANNEL PROTEIN, | increased in many | 649192 | GB | X65361 | H. sapiens | AA214661 | AA211081 |
| BRAIN I ALPHA SUBUNIT | tissues with aging | | | | | | |
| GABA-A RECEPTOR PI | increased in many | 563598 | GB | U95367 | H. sapiens | AA102670 | AA101225 |
| SUBUNIT | tissues with aging | | | | | | |
| CASEIN KINASE II BETA | increased in many | 548498 | GB | M30448 | H. sapiens | AA082829 | AA101085 |
| SUBUNIT | tissues with aging | | | | | | |
| ALPHA2-C4-ADRENERGIC | increased in many | 60664 | GB | J03853 | H. sapiens | T39448 | T40595 |
| RECEPTOR | tissues with aging | | | | | | |
| CYTOPLASMIC TYROSINE- | increased in many | 624566 | GB | X83107 | H. sapiens | AA188451 | AA187327 |
| PROTEIN KINASE BMX | tissues with aging | | | | | | |
| PROTO-ONCOGENE TYROSINE- | increased in many | 663987 | GB | X16416 | H. sapiens | AA227275 | AA227276 |
| PROTEIN KINASE ABL | tissues with aging | | | | | | |
| OSH1 PROTEIN | increased in many | 282057 | SP | P35845 | S. cerevisiae | N53619 | N51477 |
| | tissues with aging | | | | | | |
| MITOGEN ACTIVATED | increased in many | 613257 | GB | U43784 | H. sapiens | AA182510 | AA181778 |
| PROTEIN KINASE ACTIVATED | tissues with aging | | | | | | |
| PROTEIN | | | | | | | |
| VASOACTIVE INTESTIAL | increased in many | 155943 | GB | L13288 | H. sapiens | R72351 | R72302 |
| PEPTIDE RECEPTOR | tissues with aging | | | | | | |
| MUSCLE ACETYLCHOLINE | increased in many | 612253 | GB | X14830 | H. sapiens | AA211274 | |
| RECEPTOR BETA-SUBUNIT | tissues with aging | | | | | | |
| SERINE/THREONINE PROTEIN | increased in many | 127719 | GB | Y13120 | H. sapiens | R09613 | |
| KINASE MO15 | tissues with aging | | | | | | |
| VIP2 RECEPTOR | increased in many | 768352 | GB | X95097 | H. sapiens | AA495891 | AA424999 |
| | tissues with aging | | | | | | |
| CELLULAR PROTO-ONCOGENE | increased in many | 728657 | GB | U08023 | H. sapiens | AA398845 | AA435890 |
| (C-MER) | tissues with aging | | | | | | |
| FIBROBLAST GROWTH FACTOR | increased in many | 470965 | GB | M87770 | H. sapiens | AA033657 | AA032183 |
| RECEPTOR (K-SAM) | tissues with aging | | | | | | |
| DHP-SENSITIVE CALCIUM | increased in many | 344091 | GB | L07738 | H. sapiens | W73801 | W73406 |
| CHANNEL GAMMA SUBUNIT | tissues with aging | | | | | | |
| (CACNL | | | | | | | |
| 40 KDA PROTEIN KINASE | increased in many | 628355 | GB | Z11695 | H. sapiens | AA196114 | AA195999 |
| RELATED TO RAT ERK2 | tissues with aging | | | | | | |
| SERINE/THREONINE PROTEIN | increased in many | 190924 | GB | AF004849 | H. sapiens | H38252 | H37893 |
| KINASE | tissues with aging | | | | | | |
| CHLORINE CHANNEL PROTEIN | increased in many | 302996 | SP | P35526 | B. taurus | W20424 | N91135 |
| P64 | tissues with aging | | | | | | |
| LEUCOCYTE ANTIGEN CD97 | increased in many | 626826 | GB | X84700 | H. sapiens | AA190942 | AA191235 |
| | tissues with aging | | | | | | |
| SERINE/THREOMINE KINASE | increased in many | 565000 | GB | Z83868 | R. novegicus | AA121044 | AA126520 |
| MARK1 | tissues with aging | | | | | | |
| PLATELET-DERIVED GROWTH | increased in many | 70728 | GB | M21616 | H. sapiens | T47292 | T47293 |
| FACTOR (PDGF) RECEPTOR | tissues with aging | | | | | | |
| CASEIN KINASE II, ALPHA′ | increased in many | 358354 | GB | M55268 | H. sapiens | W95955 | W95869 |
| CHAIN | tissues with aging | | | | | | |
| RETINOIC ACID RECEPTOR | increased in many | 589706 | GB | Y00291 | H. sapiens | AA157597 | AA147673 |
| BETA-2 | tissues with aging | | | | | | |
| PROTEIN KINASE NEK2 | increased in many | 415089 | GB | Z29066 | H. sapiens | W94994 | W93379 |
| | tissues with aging | | | | | | |
| DNA-DEPENDENT PROTEIN | increased in many | 200884 | GB | U47077 | H. sapiens | R98882 | R98972 |
| KINASE CATALYTIC SUBUNIT | tissues with aging | | | | | | |
| (DN | | | | | | | |
| POSSIBLE GLOBAL | increased in many | 69589 | GB | U29175 | H. sapiens | T53548 | T53549 |
| TRANSCRIPTION ACTIVATOR | tissues with aging | | | | | | |
| SNF2L4 | | | | | | | |
| P58/GTA | increased in many | 429403 | GB | M37712 | H. sapiens | AA007683 | AA007684 |
| (GALACTOSYLTRANSFERASE | tissues with aging | | | | | | |
| ASSOCIATED PROTEIN | | | | | | | |
| CASEIN KINASE I, DELTA | increased in many | 284450 | GB | U29171 | H. sapiens | N75096 | N52326 |
| ISOFORM | tissues with aging | | | | | | |
| OLFACTORY RECEPTOR | increased in many | 687896 | GB | X87825 | H. sapiens | AA235800 | AA235801 |
| EXPRESSED PSEUDOGENE | tissues with aging | | | | | | |
| SRC-LIKE KINASE (SLK) | increased in many | 232949 | GB | M14676 | H. sapiens | H75607 | H74014 |
| | tissues with aging | | | | | | |
| GUANINE NUCLEOTIDE | increased in many | 429234 | GB | U42390 | H. sapiens | AA007298 | AA007299 |
| EXCHANGE FACTOR PROTEIN | tissues with aging | | | | | | |
| TRIO | | | | | | | |
| INSULIN RECEPTOR (INSR) | increased in many | 427812 | GB | X02160 | H. sapiens | AA001106 | AA001614 |
| | tissues with aging | | | | | | |
| CALMODULIN-DEPENDENT | increased in many | 430337 | GB | D14906 | O. cuniculus | AA010623 | AA010624 |
| PROTEIN KINASE II-DELTA | tissues with aging | | | | | | |
| DASH | | | | | | | |
| PROTO-ONCOGENE TYROSINE- | increased in many | 160664 | GB | X56348 | H. sapiens | H24996 | H24956 |
| PROTEIN KINASE RECEPTOR | tissues with aging | | | | | | |
| RE | | | | | | | |
| MEMBRANE-ASSOCIATED | increased in many | 730977 | GB | U56816 | H. sapiens | AA421203 | AA416587 |
| KINASE (MYT1) | tissues with aging | | | | | | |
| KIAA0151 | increased in many | 120254 | GB | D63485 | H. sapiens | T95734 | T95631 |
| | tissues with aging | | | | | | |
| G PROTEIN-COUPLED | increased in many | 119199 | GB | U21051 | H. sapiens | T94021 | T93343 |
| RECEPTOR (GPR4) | tissues with aging | | | | | | |
| ZIP-KINASE | increased in many | 154047 | GB | AB007144 | H. sapiens | R48923 | R48811 |
| | tissues with aging | | | | | | |
| GLYCINE RECEPTOR BETA | increased in many | 28471 | GB | U33267 | H. sapiens | R13383 | R40899 |
| SUBUNIT (GLRB) | tissues with aging | | | | | | |
| DUFFY BLOOD GROUP | increased in many | 181704 | GB | U01839 | H. sapiens | H39902 | H28423 |
| ANTIGEN (FYA-B+) | tissues with aging | | | | | | |
| RAC-ALPHA | increased in many | 429655 | GB | M63167 | H. sapiens | AA011602 | AA011575 |
| SERINE/THREONINE KINASE | tissues with aging | | | | | | |
| CAMP-DEPENDENT PROTEIN | increased in many | 590277 | GB | M33336 | H. sapiens | AA147595 | AA155665 |
| KINASE TYPE I-ALPHA | tissues with aging | | | | | | |
| SUBUNIT | | | | | | | |
| DUAL SPECIFICITY | increased in many | 546853 | GB | U25265 | H. sapiens | | AA083075 |
| MITOGEN-ACTIVATED | tissues with aging | | | | | | |
| PROTEIN KINASE | | | | | | | |
| PROTEIN TYROSINE KINASE | increased in many | 142994 | GB | U02680 | H. sapiens | R71154 | R71651 |
| | tissues with aging | | | | | | |
| PKU-ALPHA | increased in many | 565171 | GB | AB004884 | H. sapiens | AA126835 | AA126768 |
| | tissues with aging | | | | | | |
| CALCIUM/CALMODULIN- | increased in many | 36153 | SP | P11730 | R. norvegicus | R20608 | R46116 |
| DEPENDENT PROTEIN KINASE | tissues with aging | | | | | | |
| TYPE I | | | | | | | |
| RECEPTOR PROTEIN- | increased in many | 29543 | GB | L36642 | H. sapiens | R15219 | |
| TYROSINE KINASE (HEK11) | tissues with aging | | | | | | |
| FIBRILLARIN | increased in many | 111988 | GB | X56597 | H. sapiens | T84637 | T91403 |
| | tissues with aging | | | | | | |
| SERINE/THREONINE PROTEIN | increased in many | 624688 | GB | Y13120 | H. sapiens | AA187708 | AA181981 |
| KINASE MO15 | tissues with aging | | | | | | |
| OLFACTORY RECEPTOR | increased in many | 486540 | GB | X87825 | H. sapiens | AA043051 | AA042813 |
| EXPRESSED PSEUDOGENE | tissues with aging | | | | | | |
| MYOSIN LIGHT CHAN | increased in many | 310019 | GB | X90870 | H. sapiens | W24158 | N99150 |
| KINASE, SMOOTH MUSCLE | tissues with aging | | | | | | |
| AND NON-M | | | | | | | |
| PROBABLE G PROTEIN- | increased in many | 123666 | GB | D10923 | H. sapiens | R02739 | R02740 |
| COUPLED RECEPTOR HM74 | tissues with aging | | | | | | |
| C-FMS PROTO-ONCOGENE | increased in many | 78845 | GB | X03663 | H. sapiens | T51164 | T46880 |
| | tissues with aging | | | | | | |
| CELL DIVISION PROTEIN | increased in many | 276282 | GB | X62071 | H. sapiens | R94587 | R94588 |
| KINASE 2 (CDK2) | tissues with aging | | | | | | |
| PROTEIN KINASE C ZETA | increased in many | 586767 | GB | Z15108 | H. sapiens | AA130745 | AA130675 |
| | tissues with aging | | | | | | |
| MONOCYTE CHEMOATTRACTANT | increased in many | 430027 | GB | U03882 | H. sapiens | AA034153 | AA034154 |
| PROTEIN 1 RECEPTOR (MCP- | tissues with aging | | | | | | |
| 1 | | | | | | | |
| TYROSINE-PROTEIN KINASE | increased in many | 31577 | GB | X60957 | H. sapiens | R20966 | R42748 |
| RECEPTOR TIE-1 PRECURSOR | tissues with aging | | | | | | |
| PROTEIN KINASE DYRK2 | increased in many | 488243 | GB | Y13493 | H. sapiens | AA088610 | AA088200 |
| | tissues with aging | | | | | | |
| LIM DOMAIN KINASE 2 | increased in many | 544527 | GB | D45906 | H. sapiens | AA075098 | AA074832 |
| | tissues with aging | | | | | | |
| NEUROPEPTIDE Y RECEPTOR | increased in many | 143332 | GB | L07615 | H. sapiens | R74269 | R74183 |
| Y1 (NPYY1) | tissues with aging | | | | | | |
| EPIDERMAL GROWTH FACTOR | increased in many | 60493 | GB | X00588 | H. sapiens | T39335 | |
| RECEPTOR PRECURSOR | tissues with aging | | | | | | |
| PLATELET-DERIVED GROWTH | increased in many | 35885 | GB | M21574 | H. sapiens | R22852 | R46063 |
| FACTOR RECEPTOR ALPHA | tissues with aging | | | | | | |
| (PDG | | | | | | | |
| ACTIVIN RECEPTOR TYPE | increased in many | 69164 | GB | X77533 | H. sapiens | T54229 | T54133 |
| IIB PRECURSOR | tissues with aging | | | | | | |
| PUTATIVE G PROTEIN- | increased in many | 115277 | GB | U39827 | M. musculus | T87010 | T86932 |
| COUPLED RECEPTOR TDAG8 | tissues with aging | | | | | | |
| RHODOPSIN | increased in many | 360598 | GB | U49742 | H. sapiens | AA015774 | AA015775 |
| | tissues with aging | | | | | | |
| TYROSINE KINASE (HTK) | increased in many | 75009 | GB | U07695 | H. sapiens | T51895 | T51849 |
| | tissues with aging | | | | | | |
| PROTEIN KINASE CK1 | increased in many | 49236 | GB | X80693 | H. sapiens | H15066 | H15067 |
| | tissues with aging | | | | | | |
| CALCIUM-ACTIVATED | increased in many | 110811 | GB | U61536 | H. sapiens | T83181 | T90653 |
| POTASSIUN CHANNEL BETA | tissues with aging | | | | | | |
| SUBUNIT | | | | | | | |
| CASEIN KINASE I, GAMMA 2 | increased in many | 323094 | GB | U89896 | H. sapiens | W42531 | W42484 |
| ISOFORM | tissues with aging | | | | | | |
| PISSLRE | increased in many | 182347 | GB | X78342 | H. sapiens | H42017 | H42018 |
| | tissues with aging | | | | | | |
| SERINE/THREONINE PROTEIN | increased in many | 362359 | GB | Y10032 | H. sapiens | AA002020 | AA001901 |
| KINASE SGK | tissues with aging | | | | | | |
| NUCLEAR ORPHAN RECEPTOR | increased in many | 108892 | GB | U22662 | H. sapiens | T78977 | T78924 |
| LXR-ALPHA | tissues with aging | | | | | | |
| PML-1 | increased in many | 154763 | GB | M79462 | H. sapiens | R55395 | R55296 |
| | tissues with aging | | | | | | |
| RYANODINE RECEPTOR | increased in many | 25322 | GB | J05200 | H. sapiens | R11793 | |
| | tissues with aging | | | | | | |
| EBV-INDUCED G PROTEIN- | increased in many | 253069 | GB | L08177 | H. sapiens | H88701 | H88656 |
| COUPLED RECEPTOR 2 | tissues with aging | | | | | | |
| ERBB-2 RECEPTOR PROTEIN- | increased in many | 300383 | GB | X03363 | H. sapiens | W07477 | N75837 |
| TYROSINE KINASE | tissues with aging | | | | | | |
| PRECURSOR | | | | | | | |
| ACETYLCHOLINE RECEPTOR | increased in many | 347370 | GB | Y00762 | H. sapiens | W81677 | |
| PROTEIN, ALPHA CHAIN | tissues with aging | | | | | | |
| PRECUR | | | | | | | |
| CALCIUM CHANNEL L-TYPE | increased in many | 491064 | GB | L29536 | H. sapiens | AA136909 | AA136881 |
| ALPHA 1 SUBUNIT | tissues with aging | | | | | | |
| (CACNL1A1) | | | | | | | |
| COT PROTO-ONCOGENE | increased in many | 589054 | GB | Z14138 | H. sapiens | AA149109 | AA151281 |
| SERINE/THREONINE-PROTEIN | tissues with aging | | | | | | |
| KINASE | | | | | | | |
| SODIUM CHANNEL 2 | increased in many | 180667 | GB | U78181 | H. sapiens | R85232 | |
| (HBNAC2) | tissues with aging | | | | | | |
| MELANOCYTE STIMULATING | increased in many | 155691 | GB | X67594 | H. sapiens | R72114 | |
| HORMONE RECEPTOR | tissues with aging | | | | | | |
| MACROPHAGE INFLAMMATORY | increased in many | 80239 | GB | L10918 | H. sapiens | T65682 | T64331 |
| PROTEIN-1-ALPHA/RANTES | tissues with aging | | | | | | |
| REC | | | | | | | |
| RETINOIC ACID RECEPTOR | increased in many | 298678 | GB | M38258 | H. sapiens | W05063 | N74673 |
| GAMMA-1 | tissues with aging | | | | | | |
| RETINOIC ACID RECEPTOR | increased in many | 155460 | GB | X06614 | H. sapiens | R71970 | R71922 |
| | tissues with aging | | | | | | |
| TYROSINE KINASE RECEPTOR | increased in many | 49318 | GB | M76125 | H. sapiens | H15336 | H15718 |
| (AXL) | tissues with aging | | | | | | |

What is claimed is:

1. A method for identifying a modulator that increases expression of aging-associated genes in a cell, the method comprising:
culturing the cell in the presence of the modulator to form a first cell culture;
contacting RNA from the first cell culture with a probe which comprises a polynucleotide sequence associated with aging, wherein the polynucleotide sequence is selected from the group consisting of sequences set out in Table 1;
determining whether the amount of the probe which hybridizes to the RNA from the first cell culture is increased relative to the amount of the probe which hybridizes to RNA from a second cell culture grown in the absence of said modulator; thereby identifying the modulator.

2. The method of claim 1, further comprising:
contacting RNA from the first cell culture with a second probe which comprises a second polynucleotide sequence associated with aging, wherein the second polynucleotide sequence is selected from the group consisting of sequences set out in Table 1;
determining whether the amount of the second probe which hybridizes to the RNA from the first cell culture is increased relative to the amount of the second probe which hybridizes to RNA from a second cell culture grown in the absence of said modulator.

3. A method for identifying a modulator that decreases expression of aging-associated genes in a cell, the method comprising:
culturing the cell in the presence of the modulator to form a first cell culture;
contacting RNA from the first cell culture with a probe which comprises a polynucleotide sequence associated with aging, wherein the polynucleotide sequence is selected from the group consisting of sequences set out in Table 1;
determining whether the amount of the probe which hybridizes to the RNA from the first cell culture is decreased relative to the amount of the probe which hybridizes to RNA from a second cell culture grown in the absence of said modulator.

4. The method of claim 3, further comprising:
contacting RNA from the first cell culture with a second probe which comprises a second polynucleotide sequence associated with aging, wherein the second polynucleotide sequence is selected from the group consisting of sequences set out in Table 1;
determining whether the amount of the second probe which hybridizes to the RNA from the first cell culture is decreased relative to the amount of the second probe which hybridizes to RNA from a second cell culture grown in the absence of said modulator.

5. A method for modulating cell aging in a patient in need thereof, the method comprising administering to the patient a compound that modulates the aging of a cell.

6. A method of claim 5, wherein the modulator increases or decreases the expression of a nucleic acid sequence set out in Table 1.

7. An isolated antisense oligonucleotide derived from a nucleic acid sequence set out in Table 1.

8. An isolated gene that encodes an antisense oligonucleotide of claim 7.

9. A recombinant cell comprising the oligonucleotide of claim 7.

10. A kit for detecting aging comprising a nucleic acid probe which comprises a polynucleotide sequence from Table 1 associated with aging and a label for detecting the presence of the probe.

11. A method for modulating the aging of a cell in a patient in need thereof, the method comprising administering a compound that decreases the expression level of a nucleic acid from Table 1 whose expression is increased with aging.

12. A method for modulating the aging of a cell in a patient in need thereof, the method comprising administering a compound that increases the expression level of a nucleic acid from Table 1 whose expression level is decreased with aging.

13. An antibody specific for the detection of a protein encoded by a sequence set out in Table 1.
